(19) United States
(12) Patent Application Publication
(10) Pub. No.: US 2023/0243833 A1
(43) Pub. Date: Aug. 3, 2023

(54) METHOD AND SYSTEM FOR DIAGNOSIS AND MANAGEMENT OF GASTROESOPHAGEAL DISEASES

(71) Applicant: RJS MEDIAGNOSTIX, New York, NY (US)

(72) Inventors: Balwant Rai, Roedovre (DK); Jasdeep Kaur, Roedovre (DK)

(21) Appl. No.: 17/906,858

(22) PCT Filed: Mar. 30, 2021
(86) PCT No.: PCT/IB2021/052596

§ 371 Date: Sep. 20, 2022

(30) Foreign Application Priority Data
Mar. 30, 2020 (IN) 202011013881
Nov. 18, 2020 (IN) 202011050217

(51) Int. Cl. G01N 33/574; G01N 33/68; G01N 33/543

(52) U.S. Cl. CPC G01N 33/57446; G01N 33/6863; G01N 33/54388; G01N 2800/52

(57) ABSTRACT

Methods and systems for the diagnosis, treatment, and management of gastroesophageal diseases including gastroesophageal reflux disease, reflux laryngitis, nonerosive reflux disease and gastric cancer using saliva based biomarkers are presented herein. The biomarkers may include E-cadherin, TGF-α, EGF, IL-6, pepsin, MOB kinase activator 1A, EphA1, MOB kinase Activator 1B, integrin alpha 5, Golgi resident protein GCP 60, FR-beta, protein SEC13 homolog, epiregulin, FGF-12, inhibitor of nuclear factor kappa B kinase catalytic subunit, vimentin, annexin A1, protein S100-A6, malate dehydrogenase, proteasome activator complex subunit 2, cathepsin D light chain, MMP-1, MMP-2, MMP-3, MMP-7, MMP-8, MMP-9, MMP-10, MMP-13, MMP-14, mucin-1, alpha-1-acid glycoprotein, antithrombin, serpin peptidase inhibitor, CTSF, HMGB1, TLR7, COPS2, NT5E, TERF1, TIMP-1, TIMP-2, TIMP-4, XPNPEP2, TGFR2, SIGLEC6, CPE, GHR, GPNMB, SLAMF8, TNFRSF19, TWEAK, IFNGR1, Notch-3, TNFRSF19L, annexin A6, α-defensin-1, caveolin 1, EGF receptor, integrin beta 4, S100A6, S100A8, S100A9, and/or haptoglobin.

### CROSS REFERENCE TO RELATED APPLICATIONS

This application claims priority to Indian Patent Application No. 202011013881, entitled “Methods, Systems, and a Kit for Diagnosis, Detection, Monitoring, and Treatment of Gastroesophagal[SIC] Reflux Disease,” filed 30 Mar. 2020, and Indian Patent Application No. 202011050217, entitled “Methods, Systems, and a Kit for Diagnosis, Detection, Monitoring, and Treatment of Gastroesophagal[SIC] Reflux Disease and Gastric Cancer,” filed 18 Nov. 2020, each of which are herein incorporated by reference in their entirety.

### FIELD OF TECHNOLOGY

Disclosed are systems and methods for the diagnosis and management of gastroesophageal diseases. Specifically, the diagnosis and management of gastroesophageal reflux disease (GERD), reflux laryngitis (RL), nonerosive reflux disease (NERD), laryngopharyngeal reflux (LPR), and gastric cancer.

### BACKGROUND

Gastroesophageal diseases including gastroesophageal reflux disease (GERD), reflux laryngitis (RL), nonerosive reflux disease (NERD), laryngopharyngeal reflux (LPR) and gastric cancers impact more than 20% of adults globally (Yamasaki T, Hemond C, Eisa M, Ganocy S, Fass R. The Changing Epidemiology of Gastroesophageal Reflux Disease: Are Patients Getting Younger?. J Neurogastroenterol Motil. 2018; 24(4):559-569. doi:10.5056/jnm18140; Bray F, Ferlay J, Soerjomataram I, Siegel R L, Torre L A, Jemal A. Global Cancer Statistics 2018: GLOBOCAN estimates of incidence and mortality worldwide for 36 cancers in 185 countries. CA Cancer J Clin, in press). In the United States alone, 60-75 million adults suffer from gastroesophageal diseases, with overall spending on all gastrointestinal diseases estimated to be $142 billion per year in direct and indirect costs, 15% of which is attributable to GERD alone (Gawron A J, French D D, Pandolfino J E, Howden C W. Economic evaluations of gastroesophageal reflux disease medical management. Pharmacoeconomics. 2014; 32(8):745-758. doi:10.1007/s40273-014-0164-8).

Generally, GERD may be diagnosed based on either tissue damage or other symptoms attributable to acid reflux (Vakil N, van Zanten S V, Kahrilas P, et al. The Montreal definition and classification of gastroesophageal reflux disease: a global evidence-based consensus. Am J Gastroenterol. 2006; 101(8):1900-20). Two-thirds of those with GERD experience heartburn but exhibit a negative esophagogastroduodenoscopy (EGD) and are diagnosed with NERD, which is a manifestation of GERD. Current methods of diagnosis including, ambulatory reflux monitoring and endoscopy are invasive, expensive, and uncomfortable for the patient (Hunt R, 25 Armstrong D, Katelaris P et al: World Gastroenterology Organisation Global Guidelines: GERD global perspective on gastroesophageal reflux disease. J Clin Gastroenterol, 2017; 51(6): 467-78). Less invasive tests, such as empirical proton pump inhibitor (PPI) treatment, also known as the “PPI test,” are less sensitive and specific and thus have limited diagnostic utility (Numans M E, Lau J, de Wit N J et al: Short-term treatment with proton-pump inhibitors as a test for gastroesophageal reflux disease: A meta-analysis of diagnostic test characteristics. Ann Intern Med, 2004; 140: 518-27).

Additionally, gastroesophageal diseases may cause long term damage even in individuals who are asymptomatic. For example, left untreated, GERD can lead to serious health problems, including reflux esophagitis, strictures, swallowing difficulties, and gastric cancer (Kim J J. Upper gastrointestinal cancer and reflux disease. J Gastric Cancer. 2013; 13(2):79-85. doi:10.5230/jgc.2013.13.2.79), one of the most prevalent cancer types in the world. Although the incidence of gastric cancer is declining, the outcomes for gastric cancer patients remain dismal due to the lack of effective diagnostic technology for early detection. While surgical resection and chemotherapy have improved overall survival rates, patient prognosis remains very poor and the 5-year survival rate is less than 30% for patients with late stage gastric cancer.

There is therefore an unmet need for alternate methods of diagnosing and managing gastroesophageal diseases.

### SUMMARY

Provided herein is a non-invasive means for diagnosing and monitoring gastroesophageal disease using a specific subset of one or more biomarkers. As disclosed herein, multiple biomarkers in the bodily fluids of an individual may be quantitatively or qualitatively measured alone or in combination as a diagnostic for gastroesophageal diseases including, but not limited to, gastroesophageal reflux disease (GERD), reflux laryngitis (RL), nonerosive reflux disease (NERD), laryngopharyngeal reflux (LPR), and gastric cancer. The one or more biomarkers may also be used to monitor the progression and severity of gastroesophageal diseases, the likelihood of relapse, the effectiveness of a particular treatment in arresting or reversing the progression of these gastroesophageal diseases, the need for additional testing, and the effect of reducing or eliminating a particular treatment regimen.

In one example, a kit for determining whether a patient has a gastroesophageal disease includes a solid support on which a plurality of agents have been affixed which in combination bind to one or more biomarkers such as EGF. Biomarkers, as used herein, may include one or more of E-cadherin, TGF-α, EGF, IL-6, pepsin, MOB kinase activator 1A, EphA1, MOB kinase Activator 1B, integrin alpha 5, Golgi resident protein GCP 60, FR-beta, protein SEC13 homolog, epiregulin, FGF-12, inhibitor of nuclear factor kappa B kinase catalytic subunit, vimentin, annexin A1, protein S100-A6, malate dehydrogenase, proteasome activator complex subunit 2, cathepsin D light chain, MMP-1, MMP-2, MMP-3, MMP-7, MMP-8, MMP-9, MMP-10, MMP-13, MMP-14, mucin-1, alpha-1-acid glycoprotein, antithrombin, serpin peptidase inhibitor, CTSF, HMGB1, TLR7, COPS2, NT5E, TERF1, TIMP-1, TIMP-2, TIMP-4, XPNPEP2, TGFR2, SIGLEC6, CPE, GHR, GPNMB, SLAMF8, TNFRSF19, TWEAK, IFNGR1, Notch-3, TNFRSF19L, annexin A6, α-defensin-1, caveolin 1, EGF receptor, integrin beta 4, S100A6, S100A8, S100A9, and haptoglobin, wherein each agent binds to a different single biomarker.

In another example, provided herein is a method for differentiating between gastroesophageal diseases including (a) testing a saliva sample from an individual to obtain measured levels of one or more biomarkers selected from a group consisting of E-cadherin, TGF-α, EGF, IL-6, pepsin, MOB kinase activator 1A, EphA1, MOB kinase Activator 1B, integrin alpha 5, Golgi resident protein GCP 60, FR-beta, protein SEC13 homolog, epiregulin, FGF-12, inhibitor of nuclear factor kappa B kinase catalytic subunit, vimentin, annexin A1, protein S100-A6, malate dehydrogenase, proteasome activator complex subunit 2, cathepsin D light chain, MMP-1, MMP-2, MMP-3, MMP-7, MMP-8, MMP-9, MMP-10, MMP-13, MMP-14, mucin-1, alpha-1-acid glycoprotein, antithrombin, serpin peptidase inhibitor, CTSF, HMGB1, TLR7, COPS2, NT5E, TERF1, TIMP-1, TIMP-2, TIMP-4, XPNPEP2, TGFR2, SIGLEC6, CPE, GHR, GPNMB, SLAMF8, TNFRSF19, TWEAK, IFNGR1, Notch-3, TNFRSF19L, annexin A6, α-defensin-1, caveolin 1, EGF receptor, integrin beta 4, S100A6, S100A8, S100A9, and haptoglobin to create a biomarker profile; and (b) comparing the measured levels of the biomarker profile with reference levels for the biomarkers, wherein a decrease and/or increase in levels of the one or more biomarkers in the biomarker profile relative to the reference levels is indicative of a specific gastroesophageal disease.

In a still further example, provided herein is a method of monitoring effectiveness of a treatment for a gastroesophageal disease includes: (a) testing a first saliva sample from an individual for levels of one or more biomarkers selected from a group of biomarkers consisting of E-cadherin, TGF-α, EGF, IL-6, pepsin, MOB kinase activator 1A, EphA1, MOB kinase Activator 1B, integrin alpha 5, Golgi resident protein GCP 60, FR-beta, protein SEC13 homolog, epiregulin, FGF-12, inhibitor of nuclear factor kappa B kinase catalytic subunit, vimentin, annexin A1, protein S100-A6, malate dehydrogenase, proteasome activator complex subunit 2, cathepsin D light chain, MMP-1, MMP-2, MMP-3, MMP-7, MMP-8, MMP-9, MMP-10, MMP-13, MMP-14, mucin-1, alpha-1-acid glycoprotein, antithrombin, serpin peptidase inhibitor, CTSF, HMGB1, TLR7, COPS2, NT5E, TERF1, TIMP-1, TIMP-2, TIMP-4, XPNPEP2, TGFR2, SIGLEC6, CPE, GHR, GPNMB, SLAMF8, TNFRSF19, TWEAK, IFNGR1, Notch-3, TNFRSF19L, annexin A6, α-defensin-1, caveolin 1, EGF receptor, integrin beta 4, S100A6, S100A8, S100A9, and haptoglobin to create a first biomarker profile; (b) treating the individual for the gastroesophageal disease; (c) taking a second saliva sample from the individual at a time point after treatment for the gastroesophageal disease has begun; (d) testing the second saliva sample for levels of the one or more biomarkers to create a second biomarker profile; and (e) comparing the first biomarker profile and the second biomarker profile, wherein a decrease and/or increase in levels of the one or more biomarkers in the second biomarker profile relative to the first biomarker profile indicates that the treatment is effective.

The biomarkers may be qualitatively or quantitatively analyzed using one or more methods known to those of skill in the art. For example, in some embodiments, these biomarkers may be identified using antibody-based methods including, but not limited to, an enzyme-linked immunosorbent assay (ELISA), a radioimmunoassay (RIA), an antibody based assay, Western blot, mass spectrometry, microarray, flow cytometry, immunofluorescence, polymerase chain reaction (PCR), aptamer-based assay, immunohistochemistry, a multiplex detection assay, a lateral flow immunoassay, or exomes.

Further disclosed is a kit for diagnosing and monitoring gastroesophageal diseases and the treatment of such diseases. The kit may include (a) a composition or panel of any one or more of the above identified biomarkers; (b) a substrate for binding a biological sample isolated from a human subject suspected of having, being at risk for, or being under treatment for gastroesophageal disease; (c) an agent that binds to at least one of the biomarkers; (d) a detectable label such as one conjugated to the agent, or one conjugated to a substance that specifically binds at least one or more of the biomarkers and provides a proportional response based on the level of biomarker present. In some aspects, the kit may additionally include a reading device configured to read labels attached to the bound biomarker conjugates.

Another embodiment is directed to a kit including a plurality of reagent strips for detecting a concentration of biomarkers in oral fluid especially a saliva sample. A reagent strip includes a sample receiving portion having a detector reagent thereon. The sample receiving portion includes an indicator constructed and arranged to indicate a specific concentration of the analyte in a saliva sample. The indicators of each strip are different from indicators of other strips of the plurality of strips for detecting different concentrations the analyte. Each strip may be constructed and arranged such that when the saliva sample is introduced onto the conjugate pad, a chemical reaction occurs if the analyte is present in the saliva sample and the specific indicator (visible) will activate if a specific concentration of the analyte to categories or types or staging of disease is present within the saliva sample.

For example, the above-described kits may be capable of diagnosis and management of GERD, RL, NERD, LPR, and gastric cancer, as well as qualitative, semi-quantitative and/or quantitative analysis of biomarkers.

Embodiments of the present disclosure may be implemented in the form of a kit, method and system. In embodiments, a kit for detecting salivary biomarkers indicative of GERD, RL, NERD, LPR, and gastric cancer in a subject may include an assay or kit. In some aspects, the assay or kit may have a solid support on which one or a plurality of agents have been affixed, directly or indirectly, and which bind to one or more biomarker in a saliva sample obtained from the subject. The solid support may provided as part of an LFA, or an ELISA, or another type of assay. The agent or agents may have an affinity for one or more of the aforementioned E-cadherin, TGF-α, EGF, IL-6, pepsin, MOB kinase activator 1A, EphA1, MOB kinase Activator 1B, integrin alpha 5, Golgi resident protein GCP 60, FR-beta, protein SEC13 homolog, epiregulin, FGF-12, inhibitor of nuclear factor kappa B kinase catalytic subunit, vimentin, annexin A1, protein S100-A6, malate dehydrogenase, proteasome activator complex subunit 2, cathepsin D light chain, MMP-1, MMP-2, MMP-3, MMP-7, MMP-8, MMP-9, MMP-10, MMP-13, MMP-14, mucin-1, alpha-1-acid glycoprotein, antithrombin, serpin peptidase inhibitor, CTSF, HMGB1, TLR7, COPS2, NT5E, TERF1, TIMP-1, TIMP-2, TIMP-4, XPNPEP2, TGFR2, SIGLEC6, CPE, GHR, GPNMB, SLAMF8, TNFRSF19, TWEAK, IFNGR1, Notch-3, TNFRSF19L, annexin A6, α-defensin-1, caveolin 1, EGF receptor, integrin beta 4, S100A6, S100A8, S100A9, and haptoglobin biomarkers and each agent may bind to a different single biomarker. Additional labeled antibodies, antigens, nanoparticles, aptamers, inhibitors, substrates, cofactors, coenzymes, lectins, nucleic acids, protein A, protein G, nonbiological ligands, boronates, triazine dyes, and/or metal-ion chelates with an affinity for specific ones of the biomarkers may be utilized to enable formation of a visible complex if one or more of the biomarkers is present in the saliva sample.

Binding of biomarkers of interest may be detected by any means generally used. In some aspects, detection may occur through the appearance of a signal such as a visible complex, for example through the use of one or more indicators including, but not limited to, dye, ligands, enzymes, microparticles, quantum dots, nanocrystals, phosphors, fluorophore, and enzyme classes of labels. In some aspects, the appearance of a visible complex, such as a color change, yields a result positive for GERD, RL, NERD, LPR, and/or gastric cancer and the extent of detected binding enables levels of severity of these diseases to be determined. Detecting that a visible complex has not formed or has formed insufficiently may provide a result indicating that the subject is healthy.

One or more indicators may be used to detect binding. In some aspects, the color or wavelength of the indicator varies depending on the biomarker concentration. That is, when the biomarker concentration is different, the indicator has a different color or hue. In addition, different color rendering effects can be achieved by mixing dyes or changing the concentration of dyes.

Another embodiment is directed to a kit including a strip for immunochromatography in which a membrane pad and an absorbent pad are partially overlapped, the membrane pad including a control line including a ligand such as antibodies that specifically bind to an antigen, ligands that specifically bind to a specific receptor, DNA pairs of complementary sequences, aptamers, etc., e.g. a maleimide group or another suitable group, the control line formed parallel to and spaced apart from a test line, where the test line includes a peptide labeling substance comprising a first ligand forming one end of the peptide labeling substance and a second ligand (e.g. biotin or other suitable ligands) forming another end of the peptide labeling substance and a third ligand (e.g., a thiol group specifically binding to the first ligand or other suitable ligands) binding to the first ligand, the test line formed to have a cutoff or reference level width (e.g., E-cadherin 3.6 ng/ml) in a direction perpendicular to a flow of fluid along the strip, and a fourth ligand specifically binding to the second ligand, wherein the first and second ligands are different from each other, and the first and fourth ligands e.g. streptavidin or other suitable ligands are different from each other, wherein all ligands are different from each other. The ligands may each include a substance which very specifically binds to each other. For example, antibodies that specifically bind to an antigen, ligands which specifically bind to a specific receptor, DNA pairs of complementary sequences that hybridize specifically to each other, and the like act as ligands with each other. The meaning of the ligand used throughout this specification means substanced that specifically bind to each other as defined above, unless specifically indicated as a ligand that specifically binds to a specific receptor, aptamer, but is not limited thereto. The absorbent pad is configured to receive a sample to be tested, the sample including the fluid.

Another embodiment is directed to a method including a first step of deploying saliva collected from an individual suspected of GERD, RL, NERD, LPR, and/or gastric cancer into a membrane pad of a kit (e.g., a kit as described above) for diagnosing and managing GERD, RL, NERD, LPR, and gastric cancer in a direction toward a moisture absorption pad to pass through a test line and a control line; a second step of identifying a signal by a labeling substance at the test line and the control line; a third step of determining the individual is positive for GERD, RL, NERD, LPR, and/or gastric cancer when a signal is detected at the control line and the test line and negative when the signal is detected only at the control line. In a following step, if a signal is not detected from the control line, it is estimated that the diagnosis is not valid.

To the accomplishment of the foregoing and related ends, certain illustrative aspects of the system are described herein in connection with the following description. The features, functions, and advantages that have been discussed can be achieved independently in various embodiments of the present disclosure or may be combined in yet other embodiments, further details of which can be seen with reference to the following description and drawings. The summary is provided to introduce a selection of concepts in a simplified form that are further described below in the Detailed Description. This summary is not intended to identify key features or essential features of any subject matter described herein.

### DETAILED DESCRIPTION

The following description relates to various compositions and methods for diagnosing, monitoring, and treating gastroesophageal diseases including, but not limited to gastroesophageal reflux disease (GERD), reflux laryngitis (RL), nonerosive reflux disease (NERD), laryngopharyngeal reflux (LPR) and gastric cancer. Further described are compositions and methods for laboratory and point-of care tests for measuring biomarkers in a sample from an individual.

The biomarkers and biomarker panels described herein may be used in methods to screen subjects who are suspected of having gastroesophageal diseases; to monitor individuals who are undergoing therapies for gastroesophageal disease; to differentially diagnose disease states associated with gastroesophageal diseases; to evaluate the severity (stage) or progression of gastroesophageal disease; to identify the need for additional and/or more invasive testing; and/or to select or modify therapies or interventions for use in treating subjects with gastroesophageal disease. In some aspects, the biomarkers or biomarker panels may be useful in distinguishing malignant from benign lesions as well as differentiating a gastroesophageal disease from other diseases.

Gastroesophageal diseases may cause a variety of symptoms including, but not limited to, a burning sensation in the chest, chest pain, difficulty swallowing, regurgitation, nausea, coughing, excessive throat clearing, bitter/sour taste in the mouth, excessive mucous, breathing difficulties, choking episodes, stomach pain, difficulty swallowing and persistent vomiting. In some stages, gastroesophageal diseases may lead to bleeding in the digestive tract. The compositions and methods described herein may be used in the treatment of and for the evaluation of treatment protocols. That is, the compositions and methods may be used in the evaluation of the mitigation, amelioration, and/or stabilization of symptoms and signs, as well as a delay in the progression of symptoms and signs of gastroesophageal diseases and symptoms of such diseases.

In various embodiments described herein are methods for predicting the development of a disease in an individual, determining the prognosis of a disease in an individual, diagnosing a disease in an individual, and/or evaluating the treatment of a disease in an individual by measuring the levels of biomarkers in an individual being monitored using a biomarker panel and comparing the measured levels to reference levels of biomarkers in a normal control, a population with a gastroesophageal disease, and/or levels taken at a previous time point from the individual being monitored. In one aspect, disclosed herein is a method of diagnosing an individual with gastroesophageal disease including a specific gastroesophageal disease including taking a biological sample from the individual, measuring the levels of biomarkers in a biomarker panel, and correlating the measurement with a disease or stage of the disease. In these embodiments, to make comparisons to the subject-derived sample, the amounts of reference biomarkers are similarly calculated. In some aspects, subjects identified as having a gastroesophageal disease may receive therapeutic treatments to slow the progression of the disease or treat the disease. A disease is considered to be progressive if the amount of biomarker moves further away from a reference level over time, whereas a disease is not progressive if the amount of biomarkers remains constant over time, approaches the reference level of a healthy individual, and/or if the symptoms of the individual with the gastroesophageal disease stabilize or improve.

The use of biomarkers allows for real-time testing to predict, diagnose, and monitor disease and treatment of gastroesophageal disease. Measurement of any combination of the biomarkers described herein may be used to assemble a biomarker panel. The combination may refer to the measurement of an entire set or any subset or sub-combination of biomarkers thereof for the detection, diagnosis, prognosis, treatment, or monitoring of a gastroesophageal disease. Specifically, the detection of a plurality of biomarkers in a sample can increase the sensitivity and/or specificity of the test. Thus, in various embodiments, a biomarker panel as described herein may be used to measure 1, 2, 3, 4, 5, 6, 7, 8, 9, 10, 11, 12, 13, 14, 15, 16, 17, 18 or more biomarkers. In exemplary embodiments, a biomarker panel may measure 1, 2, 3, 4, 5, 6, or 7 biomarkers. In some embodiments, a biomarker panel may measure 1, 2, or 3 biomarkers. Relevant biomarkers for the predicting, diagnosing, monitoring, and treating gastroesophageal disease include E-cadherin, TGF-α, EGF, IL-6, pepsin, MOB kinase activator 1A, EphA1, MOB kinase activator 1B, integrin alpha 5, Golgi resident protein GCP 60, FR-beta, protein SEC13 homolog, epiregulin, FGF-12, inhibitor of nuclear factor kappa B kinase catalytic subunit, vimentin, annexin A1, protein S100-A6, malate dehydrogenase, proteasome activator complex subunit 2, cathepsin D light chain, MMP-1, MMP-2, MMP-3, MMP-7, MMP-8, MMP-9, MMP-10, MMP-13, MMP-14, mucin-1, alpha-1-acid glycoprotein, antithrombin, serpin peptidase inhibitor, CTSF, HMGB1, TLR7, COPS2, NT5E, TERF1, TIMP-1, TIMP-2, TIMP-4, XPNPEP2, TGFR2, SIGLEC6, CPE, GHR, GPNMB, SLAMF8, TNFRSF19, TWEAK, IFNGR1, Notch-3, TNFRSF19L, annexin A6, α-defensin-1, caveolin 1, EGF receptor, integrin beta 4, S100A6, S100A8, S100A9, and haptoglobin. In some embodiments, the biomarkers may be one or more of E-cadherin, TGF-α, EGF, IL-6, MMP-2, MMP-7, and pepsin. In some embodiments, a biomarker panel may be used to measure levels of E-cadherin, EGF, and pepsin; E-cadherin and pepsin; EGF and pepsin; E-cadherin and EGF; E-cadherin, EGF, IL-6, MMP-7, and pepsin; E-cadherin, EGF, MMP-2, MMP-7, and pepsin; or EGF, IL-6, MMP-2, MMP-7, pepsin, and MMP-8.

In further embodiments a biomarker panel may be used to measure levels of E-cadherin, EGF, and/or pepsin. In another embodiment, a biomarker panel may be used to measure levels of E-cadherin, EGF, or pepsin and MMP-7 or MMP-9. In an additional embodiment, a biomarker panel may be used to measure levels of E-cadherin or EGF and one or more additional biomarkers. In some embodiments, the biomarkers may be one or more of E-cadherin, TGF-α, EGF, IL-6, MMP-2, MMP-7, pepsin, MMP-1, MPP-2, MMP-8, MMP-9, MMP-10, MMP-13, TIMP-1, TIMP-2, S100A8, S100A9, and haptoglobin. In some embodiments a single biomarker may be measured. In other embodiments a biomarker panel may be used to measure one or more of the listed biomarkers in any combination. It will be appreciated that the specific identity of biomarkers within the panel and the number of distinct biomarkers within the panel can depend on the particular use to which the biomarker panel is put and the stringency that the results of panel must meet for the particular application. For example, a biomarker panel for GERD may include one or more of salivary E-cadherin, TGF-α, EGF, IL-6, MMP-2, MMP-7, and pepsin with specificity/sensitivity in a range of 0.69 to 1/0.70-0.99. A biomarker panel for RL may include one or more of salivary E-cadherin, EGF, MMP-2, MMP-7, and pepsin with specificity/sensitivity in a range of 0.70 to 0.99/0.69-0.97. A biomarker panel for NERD may include one or more of salivary E-cadherin, TGF-α, EGF, IL-6, MMP-2, MMP-7, and pepsin with specificity/sensitivity in a range of 0.70 to 0.99/0.69-0.99. A biomarker panel for LPR may include one or more of salivary E-cadherin, EGF, IL-6, MMP-2, MMP-7, and pepsin with specificity/sensitivity in a range of 0.65 to 0.99/0.76-0.99. A biomarker panel for gastric cancer may include one or more of salivary E-cadherin, EGF, MMP-2, MMP-7, and pepsin with specificity/sensitivity in a range of 0.75 to 0.99/0.75-0.99.

Biomarkers may be collected using any means generally used from any bodily fluid including, but not limited to, saliva, blood, gingival crevicular fluid, serum, plasma, urine, nasal swab, cerebrospinal fluid, pleural fluid, synovial fluid, gastric fluid, peritoneal fluid, lymph fluid, interstitial fluid, tissue homogenate, cell extracts, saliva, sputum, stool, physiological secretions, tears, mucus, sweat, fluid from ulcers and other surface eruptions, blisters, and abscesses, and extracts of tissues including biopsies of normal, and suspect tissues or any other constituents of the body which may contain the target molecule of interest. However, generally the biomarkers may be identified from saliva.

Saliva samples may be collected using the same, different, or similar types of collection methods to those used for prior collections and/or in comparison to collection methods used for reference levels. As different types of collection methods may yield different biomarker levels, in some embodiments, all saliva samples may be collected in the same manner. For example, the original sample and repeat samples may be collected using the same type of collection method used in determining the reference levels. Saliva may be collected using stimulated and unstimulated means. As an example, the saliva samples may be collected via a device wherein the individuals position their tongues on the lingual surfaces of the upper incisors, lean forward, and allow their saliva to drip into the device. In other examples, saliva may be collected using an absorbent pad material. “Reference levels” may be an absolute value, a relative value, a value that has an upper and/or lower limit, a range of values, an average value, a median value, a mean value, a shrunken centroid value, a value as compared to a particular control or baseline value or a combination thereof. It is to be understood that other statistical variables may be used in determining the reference value.

Protein/biomarker levels in saliva of human subjects may vary based on various factors, such as age and gender (Bhuptani D, Kumar S, Vats M, Sagav R. Age and gender related changes of salivary total protein levels for forensic application. J Forensic Odontostomatol. 2018 May 30; 36(1):26-33. PMID: 29864027; PMCID: PMC6195944). Thus, in some aspects, saliva samples taken from individuals for sample testing, reference level determination, and the like may be collected from similar types of individuals in a same manner, including in the same time of day window, with the same saliva collection method, etc., to maintain consistency in the samples. As used herein, the term “time of day window” when referring to times that samples are taken means a period of time defined by a window start time and a window stop time. The terms “time of day window”, “window start time”, and “window stop time” all refer to local times where a sample was taken. The phrase “same time of day window” when referring to samples taken from multiple subjects means the same time of day window in local time, regardless of the time zone in which the sample is taken. For example, if one test subject is tested at 10:15 am local time in the +5 time zone and on the same day another subject is tested at 10:15 am local time in the −8 time zone, both samples are considered to have been taken in the same time of day window of 10:00-11:00 am, even though by Coordinated Universal Time, they were taken about 13 hours apart. Likewise, the samples taken in the foregoing example would be considered “within the same time of day window” even if they were taken at those times on different calendar days.

The biomarkers used herein to predict, diagnose, or monitor gastroesophageal disease may be measured using any process known to those of skill in the art including, but not limited to, enzyme linked immunosorbent assay (ELISA), fluorescence polarization immunoassay (FPIA) and homogeneous immunoassays, point of care tests using conventional lateral flow immunochromatography (LFA), quantitative point of care tests using determination of chemiluminescence, fluorescence, and magnetic particles, as well as latex agglutination, biosensors, gel electrophoresis, mass spectrometry (MS), gas chromatograph-mass spectrometry (GC-MS), and nanotechnology based methods, by way of example. This technology includes qualitative or quantitative measurement of levels of gastroesophageal disease biomarkers in a biological sample such as saliva. Such technologies include immunofluorescent assays, enzyme immunoassays, radioimmunoassays, chemiluminescent assays, sandwich-format assays, techniques using microfluidic or MEMS technologies, re-engineering technologies (e.g. instruments utilizing sensors for biomarkers used for telemedicine purposes), epitope-based technologies, other fluorescence technologies, microarrays, lab-on-a-chip, and rapid point-of-care screening technologies. For example, as shown in Example 1, below, biomarkers may be identified using an ELISA test specific for the biomarker(s) of interest which generates a color change that can be measured using a spectrophotometer.

In some embodiments, biomarker assays may be pretreated with magnetic particles, nanoparticles, or one of a series of other molecules coated with a substance or molecule capable of specifically or non-specifically binding to components in the saliva thereby improving the performance of the assay by blocking or obstructing interfering substances.

The biomarkers of the invention show a statistically significant difference in individuals with a gastroesophageal disease and healthy controls and between GERD and gastric cancer as shown in Tables 1, 4, and 9, below. In various embodiments, diagnostic tests that use these biomarkers alone or in combination show a sensitivity and specificity of at least about 80%, at least about 82%, at least about 85%, at least about 89%, at least about 90%, at least about 92%, at least about 94%, at least about 95%, at least about 96%, and about 100%. For example, the combination of the biomarkers E-cadherin and EGF have been unexpectedly found to be highly predictive of GERD, LPR, RL, NERD, and gastric cancer in the broadest possible age range of subjects from children through older populations within just a few minutes. Furthermore, the combinations of E-cadherin and MMP-7, E-cadherin and pepsin, EGF and pepsin, and pepsin and MMP-7 and MMP-9 have been unexpectedly found to be highly predictive in a rapid diagnostic of GERD, LPR, RL, NERD and gastric cancer in the broadest possible age range of subjects from children through older people. That is, the combination of these biomarkers have been found to be highly predictive within minutes.

Biomarkers and biomarker panels as described herein may be used alone or in combination with other diagnostic tools including, but not limited to, a combination of one or more of esophagogastroduodenoscopy, endoscopic ultrasound, signs and symptoms, upper gastrointestinal tract series i.e. barium swallow, endoscopic retrograde cholangiopancreatography, pH monitoring, esophageal/gastric manometry, flexible sigmoidoscopy, virtual colonoscopy, capsule endoscopy, anorectal manometry, fecal blood test, breath test, magnetic resonance imaging, different gastric examination scales, gastric juice examinations, etc. In some aspects, the biomarker panels may be used to determine the need for additional testing.

In some embodiments, the levels of the biomarkers may be assessed using a lateral flow device. In some lateral flow devices, one or two or more than two lateral flow test strips are present together in a single housing or cassette. In some embodiments, the lateral flow device further includes a reservoir containing an amount of specific buffer sufficient for the assay to properly function if the specific buffer is loaded on the sample pad of each of the one or two or more than two test strips after exposing the strip(s) to the subject's sample, for example saliva. While different assays may use different amounts of saliva, in some embodiments, the patient saliva sample is 1 uL to 200 uL.

In other embodiments, the level of biomarkers may be assessed using a nanozyme-enabled assay. For example, a nanozyme assay may be prepared using Fe—N—C single-atom derived from Fe-doped polypyrrole nanotube, which may be substituted for typical colormetric/visualization enzymes such as horseradish peroxidase on the agent(s) in the kit, which may enhance the detection sensitivity of the biomarkers.

The assays and methods described herein may be used to test any sample volume generally tested. In some aspects, the methods described herein may be used to test small sample volumes (e.g., about 0.001, 0.005, 0.01, 0.02, 0.01, 0.1, 0.5, 1, 2, 3, 4, 5, 6, 7, 8, 9, 10 l) with high sensitivity. For example, the level of biomarkers may be assessed using an antibody micropatterned lubricant infused assay capable of detecting levels as low as 0.001 picogram of biomarkers in human fluids, especially saliva.

In some embodiments, biomarkers may be bound using nanozymes such as the single atom Fe—Nx which exhibits peroxidase-mimicking activity. In some aspects, a binding agent, e.g., antibody or aptamer, might be immobilized on encoded particles such as a Fe—Nx single-atom. With bound biomarkers on the capture reagent on the encoded particles, the identity of the bound biomarkers can be detected with detection antibodies or aptamers.

The methods used herein include establishing reference levels for biomarkers. The reference levels may be established from individuals who do not have a gastroesophageal disease, individuals who do have a gastroesophageal disease, or taken at a different point in time from the individual of interest undergoing testing. These reference levels may be used as a comparison for biomarker levels in samples (such as saliva) obtained from an individual at risk for a gastroesophageal disease, suspected of having a gastroesophageal disease, or under treatment for a gastroesophageal disease. In some examples, the control is a sample from a subject not known to have a gastroesophageal disease. The individual is determined to have a gastroesophageal disease if the biomarker levels are statistically different in relative amounts to the biomarkers in the biological sample of a healthy control. As an example, biomarker levels in healthy control subjects may be measured, and a mean level and standard deviation calculated for each biomarker. The individual may be determined to the gastroesophageal disease if one or more of the individual's measured biomarker is statistically different than the corresponding healthy control level for that biomarker(s), which may include being less than or greater than mean level, being less than or greater than a range encompassed by the standard deviations (e.g., the mean plus the standard deviation and the mean minus the standard deviation), and/or being less than or greater than a threshold range encompassing the mean (e.g., the mean plus and minus 10% of the mean). The individual is determined to have a gastroesophageal disease if the biomarker levels are statistically equivalent to the biomarker levels in a population previously diagnosed with a gastroesophageal disease. The individual is determined to be responding to treatment for a gastroesophageal disease if the relative amounts of the biomarkers in the biological sample have altered from the biomarkers in a biological sample taken at an earlier first time point from the individual. The disease state of the individual may be progressing if the biomarker levels in a biological fluid are changing relative to the levels in the individual taken at an earlier time point. Various reference levels, target levels, and ranges are described herein. It is to be understood that when biomarker levels are compared to a respective reference level (e.g., a mean), the reference level may include a range around the reference level, such as plus and minus 10% of the reference level or plus and minus the standard deviation.

In some embodiments, the measured value of an individual suspected of having a gastroesophageal disease, or being treated for a gastroesophageal disease, may be diagnosed by calculating the number of fold differences (i.e. 2-fold, 3-fold, etc.) between the measured value in the individual and the reference value. A fold difference can additionally be a value in the range of 10% to 90% of the reference value. In other embodiments, a fold difference can be determined by measuring the absolute concentration of a biomarker and comparing that to the absolute value of a reference. Alternately, a fold difference can be measured as the relative difference between a reference value and a sample value, where neither value is a measure of absolute concentration, and/or where both values are measured simultaneously. In other embodiments, the fold difference between the measured value in the individual and the reference value may be compared to a minimum fold difference. In additional embodiments, the measured levels in a particular individual may be normalized against values from normal, healthy individuals. In further embodiments, the measured levels for a particular individual may be compared to reference levels for healthy individuals and reference levels of individuals previously diagnosed with a gastroesophageal disease. In another embodiment, the reference levels for biomarkers are established based on biomarker levels in a sample taken from a subject at a previous point in time. The subject is estimated to be reacting to treatment, intervention, and management for a gastroesophageal disease (e.g., GERD, LPR, RL, NERD and/or gastric cancer) if levels of the biomarkers in the biological sample, especially in saliva, have changed (e.g., increased or decreased) from the biomarker levels in a biological sample taken at an earlier time point from the same subject. For example, a decrease in the levels of some biomarkers, e.g., E-cadherin, TGF-α, EGF, IL-6, MMP-2, MMP-7, pepsin, MMP-1, MMP-2, MMP-3, MMP-8, MMP-14, MMP-9, MMP-10, MMP-13, S100A8, S100A9, and/or haptoglobin, may indicate that the treatment is effective while an increase in the levels of other biomarkers, e.g., TIMP-1 and TIMP-2, may also indicate that the treatment is effective. In another embodiment, the reference levels for biomarkers are established based on biomarker levels in a sample taken from a subject at a previous point in time. The subject is estimated to be reacting to treatment, intervention, and management for a gastroesophageal disease (e.g., GERD, LPR, RL, NERD and/or gastric cancer) if levels of the biomarkers in the biological sample, especially in saliva, have changed (e.g., increased or decreased) from the biomarker levels in a biological sample taken at an earlier time point from the same subject.

As shown in Table 1 of Example 1, below, different levels of salivary E-cadherin, EGF, TGF-α, pepsin, MMP-7, and MMP-2 biomarkers were demonstrated to be useful in discriminating GERD patients from controls. Salivary E-cadherin, TGF-α, EGF, IL-6, MMP-2, MMP-7, pepsin, MMP-1, MMP-2, MMP-3, MMP-8, MMP-14, MMP-9, MMP-10, MMP-13, TIMP-1, TIMP-2, S100A8, S100A9 and haptoglobin biomarkers were also demonstrated to be useful in discriminating between gastric cancer, GERD, and controls as shown in Table 4 and Table 9, below, with sensitivities and specificities of the individual biomarkers reaching as high as 82% for individual biomarkers (Table 5) and 99% sensitivity and 99% specificity for combinations of biomarkers (Table 5). Additionally, the data was reproducible as shown in Tables 10 and 11. Therefore salivary biomarkers, and specifically E-cadherin, TGF-α, EGF, IL-6, MMP-2, MMP-7, pepsin, MMP-1, MMP-2, MMP-3, MMP-8, MMP-14, MMP-9, MMP-10, MMP-13, TIMP-1, TIMP-2, S100A8, S100A9 and haptoglobin biomarkers are effective in predicting, diagnosing and monitoring of gastroesophageal disease.

Additionally, as shown in Tables 1, 4, and 9, below, salivary E-cadherin, TGF-α, EGF, IL-6, MMP-2, MMP-7, pepsin, MMP-1, MMP-2, MMP-3, MMP-8, MMP-14, MMP-9, MMP-10, MMP-13, TIMP-1, TIMP-2, S100A8, S100A9 and haptoglobin biomarkers are useful in discriminating between GERD and gastric cancer. For example, reference levels for a diagnosis of GERD may include: 9.4-14.5 ng/mL of E-cadherin, 1482.0-2085.0 pg/mL of EGF, 21.8-31.8 pg/mL of TGF-α, 137.4-180.0 ng/mL of pepsin, 881.1-1169.8 pg/mL of MMP-7, 12.5-32.1 pg/mL of MMP-2, 87.4-125 pg/mL of MMP-1, 154.7-201.9 ng/mL of MMP-3, 375.8-471.4 ng/ML of MMP-8, 154.1-203.7 ng/mL of MMP-9, 110.7-180.3 pg/mL of MMP-10, 0.08-0.3 ng/mL of MMP-13, 234.8-300 pg/mL of TIMP-1, 135-221.8 pg/mL of TIMP-2, 0.2-1.2 pg/mL of S100A8, 130.9-198.1 ng/mL of S100A9, and 12.8-36.8 μg/mL of haptoglobin. Reference levels may additionally be smaller subsets of these ranges or threshold levels. An individual may be diagnosed as having GERD if their biomarker levels fall within one or more of these ranges.

Reference levels for a diagnosis of gastric cancer may include: 18.8-23.3 ng/mL of E-cadherin, 2020.1-2449.3 pg/mL of EGF, 43.9-64.7 pg/mL of TGF-α, 194.4-225 ng/mL of pepsin, 1594.6-1870.2 pg/mL of MMP-7, 27.9-41.1 pg/mL of MMP-2, 232.9-288.7 pg/mL of MMP-1, 246.4-311.4 ng/mL of MMP-3, 616.8-714 ng/ML of MMP-8, 254.7-328.1 ng/mL of MMP-9, 214.2-296.6 pg/mL of MMP-10, 1.01-2.29 ng/mL of MMP-13, 76.9-144.3 pg/mL of TIMP-1, 84.3-130.7 pg/mL of TIMP-2, 3-6.2 pg/mL of S100A8, 297.9-348.9 ng/mL of S100A9, and 48.1-74.7 μg/mL of haptoglobin. Reference levels may additionally be smaller subsets of these ranges or threshold levels. For example, an individual may be diagnosed as having gastric cancer if their biomarker levels fall within one or more of these ranges, or if their biomarker levels are above the lower value of one or more of these ranges.

If a subject has any two or more salivary biomarkers that are at certain levels, such as E-cadherin ≥5 ng/ml, EGF≥978 pg/mL, TGF-α≥19.2 pg/mL, pepsin ≥92.4 ng/mL, MMP-7≥724 pg/mL, MMP-2≥12.4 pg/mL, MMP-1≥78 pg/ml, MMP-3≥100 ng/ml, MMP-8≥245 ng/mL, MMP-9≥95 ng/ml, MMP-10≥90 pg/ml, MMP-1 3≥0.05 ng/ml, S100A8≥0.4 pg/ml, S100A9≥91 ng/ml, and/or haptoglobin ≥15 μg/mL, while TIMP-1≤291 pg/ml and/or TIMP-2≤190 pg/ml, the subject may be recommended for additional testing, for example gastrointestinal endoscopy examination. In particular, if the subject has levels of E-cadherin ≥5 ng/ml, EGF≥978 pg/mL, and pepsin ≥92.4 ng/mL, the subject may be recommended for an endoscopy. In some examples, if a subject has any two or more salivary biomarkers that are at certain levels, such as within plus and minus 10% of a respective target value, the subject may be recommended for gastrointestinal endoscopy examination. In some examples, the target values may include E-cadherin 5 ng/ml, EGF 978 pg/mL, TGF-α 19.2 pg/mL, pepsin 92.4 ng/mL, MMP-7 724 pg/mL, MMP-2 12.4 pg/mL, MMP-1 78 pg/ml, MMP-3 100 ng/ml, MMP-8 245 ng/mL, MMP-9 95 ng/ml, MMP-10 90 pg/ml, MMP-1 3 0.05 ng/ml, S100A8 0.4 pg/ml, S100A9 91 ng/ml, haptoglobin 15 μg/mL, TIMP-1 291 pg/ml, and/or TIMP-2 190 pg/ml.

A method for assessing gastroesophageal health of a human to determine the need for or effectiveness of a treatment regimen includes: a) obtaining a biological sample, such as saliva, from a human; b) determining, prior to treatment, levels of one or more of, and in some examples at least two, biomarkers selected from the group consisting of E-cadherin, TGF-α, Epidermal Growth Factor (EGF), Interleukin-6 (TL-6), pepsin, MOB kinase activator 1A, EphA1, MOB kinase Activator 1B, integrin alpha 5, Golgi resident protein GCP 60, FR-beta, protein SEC13 homolog, Epiregulin, FGF-12, Inhibitor of nuclear factor kappa B kinase catalytic subunit, vimentin, annexin A1, protein S100-A6, Malate dehydrogenase, Proteasome activator complex subunit 2, Cathepsin D light chain, MMP-1, mucin-1, alpha-1-acid glycoprotein, MMP-2, Antithrombin, serpin peptidase inhibitor, MMP-3, CTSF, MMP-8, HMGB1, MMP14, TLR7, COPS2, NT5E, 20 MMP-9, MMP-10, TERF1, MMP-13, TIMP-1, TIMP-2, TIMP-4, XPNPEP2, TGFR2, SIGLEC6, CPE, GHR, GPNMB, SLAMF8, TNFRSF19, TWEAK, IFNGR1, Notch-3, TNFRSF19L, annexin A6, α-defensin-1, caveolin 1, epidermal growth factor receptor, integrin beta 4, S100A6, S100A8, S100A9, haptoglobin, MMP-7, and MMP-2 in the biological sample; c) obtaining a reference dataset comprised of the levels of each biomarker; d) inputting the reference dataset and the determined levels of the one or more biomarkers into an analytical classification process that uses the determined levels of the one or more biomarkers and the reference dataset to classify the biological sample into a classification, wherein the classification is selected from the group consisting of mild, moderate, severe classifications of each of GERD, LPR, RL and NERD, different staging of gastric cancer, a healthy classification, a medication exposure classification, and a no medication exposure classification prior to treatment; and e) determining a treatment regimen for the human based on the classification.

Biomarkers may be measured individually or as part of a panel of biomarkers. In some embodiments, biomarkers for gastroesophageal disease as disclosed herein are attached to a surface such that levels might be obtained directly or indirectly. In further embodiments, gastroesophageal disease biomarker-specific affinity reagents are bound to a solid support to provide separation of the biomarkers in biological samples particularly saliva. Biomarker complex formation leads to at least one gastroesophageal disease diagnostic biomarker bound to a reagent specific for the biomarker, wherein said biomarker is attached to a surface. Binding or complex formation can be estimated qualitatively or quantitatively. Both standard and competitive formats for these assays including point of care systems are known in the art. The bound biomarkers are detected using a mixture of appropriate detection reagents, which includes fluorescent dye-based or other visual systems that specifically bind various biomarkers.

In a further embodiment, the present invention may be a biochip assay, a composition generally comprising a solid support or substrate to which a capture binding ligand is attached and can bind to proteins. Detection of a target species in some embodiments requires a label or detectable marker that can be incorporated as generally known to those of skill in the art. Such labels may be isotopic labels; magnetic, electrical or thermal labels; colored or luminescent dye; and enzymes, all of which enable detection of the biomarkers. In various embodiments, a secondary detectable label is used. A secondary label is one that is indirectly detected including, but not limited to, one of a binding partner pair; chemically modifiable moieties; nuclease inhibitors; enzymes such as horseradish peroxidase, alkaline phosphatases, luciferases etc. In sandwich formats of the invention, an enzyme serves as the secondary label, bound to the soluble capture ligand. In various embodiments, the system relies on detecting the precipitation of a reaction product or on a change on the properties of the label, for example the color for detection. A detection system for colorimetric methods includes any device that can be used to measure colorimetric properties. Generally, the device is a spectrophotometer, a colorimeter, or any device that measures absorbance or transmission of light on one or more wavelengths.

In one embodiment, this disclosure provides a kit or kits for the diagnosis, monitoring, prognosis, treatment, and detection of a gastroesophageal disease (including GERD, LPR, RL, NERD, and/or gastric cancer). The kit may include: (a) a panel of any one or two, more than two, all, or more of the above-identified biomarkers such as E-cadherin, TGF-α, Epidermal Growth Factor (EGF), Interleukin-6 (IL-6), pepsin, MOB kinase activator 1A, EphA1, MOB kinase Activator 1B, integrin alpha 5, Golgi resident protein GCP 60, FR-beta, protein SEC13 homolog, epiregulin, FGF-12, Inhibitor of nuclear factor kappa B kinase catalytic subunit, vimentin, annexin A1, protein S100-A6, Malate dehydrogenase, Proteasome activator complex subunit 2, Cathepsin D light chain, MMP-1, mucin-1, alpha-1-acid glycoprotein, MMP-2, Antithrombin, serpin peptidase inhibitor, MMP-3, CTSF, MMP-8, HMGB1, MMP14, TLR7, COPS2, NT5E, MMP-9, MMP-10, TERF1, MMP-13, TIMP-1, TIMP-2, TIMP-4, XPNPEP2, TGFR2, SIGLEC6, CPE, GHR, GPNMB, SLAMF8, TNFRSF19, TWEAK, IFNGR1, Notch-3, TNFRSF19L, annexin A6, α-defensin-1, caveolin 1, epidermal growth factor receptor, integrin beta 4, S100A6, S100A8, S100A9, haptoglobin, MMP-7, and MMP-2; (b) a substrate for holding a biological sample isolated from a human subject suspected of having the gastroesophageal disease, or being under treatment or intervention for the gastroesophageal disease; (c) an agent which connects or binds to at least one of the biomarkers; (d) a measurable label, e.g., one conjugated to the agent, or one conjugated to a substance which specially binds at least to one or more of the biomarkers and presents a proportional reaction based on the level of biomarker present; and (e) printed or computer based, or e-printed, or remote instructions for reacting the agent with the biological sample, or a portion of the biological sample, to detect the presence or concentration of at least one biomarker in the biological sample and estimate if the biomarker is within a reference level of the biomarker. In some aspects, the kit may additionally include a measurement device operable to indicate the measurable label to provide a qualitative or quantitative level of one or more biomarkers in the saliva sample. In another embodiment, the kit may include a dataset comprised of the levels of each biomarker which can be used to classify the biological sample, wherein the classification is selected from the group consisting of mild, moderate, and severe classifications of each of GERD, LPR, RL and NERD, different staging of gastric cancer, a healthy classification, a medication exposure classification, and a no medication exposure classification; and a treatment regime based on the classification.

Labels which generate a measureable signal relating to the presence or absence of a biomarker or set of biomarkers may include, but are not limited to, fluorescent dyes, chemiluminescent compounds, radioisotopes, electron-dense reagents, enzymes, or colored particles, nanoparticles, metal sol or colloid, gold or silver nanoparticle nanoparticles, colored latex beads, up-converting phosphors, magnetic particles, carbon nanoparticles, selenium nanoparticles, quantum dots, organic fluorophores, and other suitable labels. The label component can generate a measurable signal, such as radioactivity, fluorescent light, color, or enzyme activity. In some examples, different biomarkers may be bound to different label components.

In some embodiments, a suitable label depends on the intended detection methods. For example, the label can be a direct label or an indirect label. A direct label can be detected by an instrument, device or naked eyes without further step to generate a detectable signal. A visual direct label, e.g., a gold or latex, sliver nanoparticle label, can be detected by naked eyes. An indirect label, e.g., an enzyme label, requires a further step to generate a detectable signal which can be detected by suitable methods, including but not limited to an optical detector (e.g., a camera configured to image fluorescent light or another type of light) or other reader, artificial intelligence, and machine learning based methods.

For example, a kit may comprise a saliva sample obtained from the patient; a plurality of test strips, each configured to produce a fluorescence level proportional to a level present on the test strip of one of a group of biomarkers; and a reading device configured to read the fluorescence levels on each of the test strips after the test strips are exposed to the saliva sample and wherein when the fluorescence levels indicate that two or more of the biomarkers are different than a respective reference level, the patient is determined to have gastroesophageal disease. In some embodiments, the kit may be used within the same time of day window, in the same manner and/or with the same test used to determine the reference levels.

The methods and compositions described herein may additionally be used to monitor the effectiveness of a treatment regime. Effectiveness may be identified as the mitigation, amelioration, and/or stabilization of symptoms and signs, as well as a delay in the progression of symptoms and signs of gastroesophageal diseases and symptoms of such diseases. Treatments for gastroesophageal diseases include, but are not limited to, one or more of antacids, H-2 receptor blockers, proton pump inhibitors, surgery, transoral incisionless fundoplication, LINX device, fundoplication, prokinetics, acupuncture, surgical intervention, resection, and chemotherapy. As explained above, certain methods for diagnosing gastroesophageal diseases, such as endoscopy, may be invasive and thus patients who exhibit mild symptoms may not be tested. The use of the biomarkers to diagnose gastroesophageal disease as described herein may facilitate earlier recognition of gastroesophageal disease (e.g., NERD or ERD) in patients with milder symptoms, allowing treatment to be administered and potentially reduce the risk that a more severe gastroesophageal disease (e.g., gastric cancer) may develop. Further, some reflux conditions, such as LPR, may be referred to as silent reflux, and as such may not exhibit classic reflux symptoms. Without reflux symptoms, a patient may not be aware that they are suffering from LPR and/or a clinician may not opt to subject the patient to invasive diagnostic methods such as endoscopy. However, the biomarkers and kits, systems, and methods disclosed herein for diagnosing gastroesophageal disease may allow for recognition of LPR without invasive diagnostic methods, which may allow for treatment before LPR advances to more a serious gastroesophageal disease. Additionally, some clinicians and/or insurers may desire to limit the frequency a patient diagnosed with, suspected of having, or predisposed to having a gastroesophageal disease is administered an endoscopy. The biomarkers and kits, systems, and methods disclosed herein for diagnosing/monitoring gastroesophageal disease may inform on how often and/or when a patient should undergo endoscopy.

The disclosure also provides support for a kit for determining whether a patient has a gastroesophageal disease, comprising: a solid support on which a plurality of agents have been affixed which in combination bind to EGF. In a first example of the kit, the plurality of agents in combination bind to one or more additional biomarkers selected from a group of biomarkers consisting of E-cadherin, TGF-α, IL-6, pepsin, MOB kinase activator 1A, EphA1, MOB kinase Activator 1B, integrin alpha 5, Golgi resident protein GCP 60, FR-beta, protein SEC13 homolog, epiregulin, FGF-12, inhibitor of nuclear factor kappa B kinase catalytic subunit, vimentin, annexin A1, protein S100-A6, malate dehydrogenase, proteasome activator complex subunit 2, cathepsin D light chain, MMP-1, MMP-2, MMP-3, MMP-7, MMP-8, MMP-9, MMP-10, MMP-13, MMP-14, mucin-1, alpha-1-acid glycoprotein, antithrombin, serpin peptidase inhibitor, CTSF, HMGB1, TLR7, COPS2, NT5E, TERF1, TIMP-1, TIMP-2, TIMP-4, XPNPEP2, TGFR2, SIGLEC6, CPE, GHR, GPNMB, SLAMF8, TNFRSF19, TWEAK, IFNGR1, Notch-3, TNFRSF19L, annexin A6, α-defensin-1, caveolin 1, EGF receptor, integrin beta 4, S100A6, S100A8, S100A9, and haptoglobin, wherein each agent binds to a different single biomarker. In a second example of the kit, optionally including the first example, the plurality of agents in combination bind to E-cadherin and EGF. In a third example of the kit, optionally including one or both of the first and second examples, the plurality of agents in combination bind to E-cadherin, EGF, and one or more of TGF-α, IL-6, MMP-2, MMP-7, and pepsin. In a fourth example of the kit, optionally including one or more or each of the first through third examples, the plurality of agents in combination bind to EGF and pepsin. In a fifth example of the kit, optionally including one or more or each of the first through fourth examples, the solid support includes a plurality of test strips, each configured to produce a detectable output at a level proportional to a level present on the test strip of EGF and the one or more additional biomarkers after the test strips are exposed to a saliva sample, and wherein when the detectable output levels indicate that EGF and/or the one or more of the biomarkers meet one or more criteria in a group of criteria, the gastroesophageal disease is indicated. In a sixth example of the kit, optionally including one or more or each of the first through fifth examples, the group of criteria include respective levels of EGF and/or the one or more biomarkers being greater than corresponding reference levels of EGF and/or the one or more biomarkers. In a seventh example of the kit, optionally including one or more or each of the first through sixth examples, the kit is for determining gastroesophageal reflux disease (GERD), laryngopharyngeal reflux (LPR), reflux laryngitis (RL), nonerosive reflux disease (NERD), and/or gastric cancer. In an eighth example of the kit, optionally including one or more or each of the first through seventh examples, wherein the plurality of agents comprise one or more of antibodies, antigens, nanoparticles, aptamers, inhibitors, substrates, cofactors, coenzymes, lectins, nucleic acids, protein A, protein G, nonbiological ligands, boronates, triazine dyes, and metal-ion chelates. In a ninth example of the kit, optionally including one or more or each of the first through eighth examples, the plurality of agents comprise one or more ligands. In a tenth example of the kit, optionally including one or more or each of the first through ninth examples, the kit is an antibody micropatterned lubricant infused kit. In an eleventh example of the kit, optionally including one or more or each of the first through tenth examples, the kit is an Fe—N—C single-atom nanozymes (SANs) based kit.

The disclosure also provides support for a method for differentiating between gastroesophageal diseases, comprising: (a) testing a saliva sample from an individual to obtain measured levels of one or more biomarkers selected from a group consisting of E-cadherin, TGF-α, EGF, TL-6, pepsin, MOB kinase activator 1a, EphA1, MOB kinase activator 1B, integrin alpha 5, Golgi resident protein GCP 60, FR-beta, protein SEC13 homolog, epiregulin, FGF-12, inhibitor of nuclear factor kappa B kinase catalytic subunit, vimentin, annexin A1, protein S100-A6, malate dehydrogenase, proteasome activator complex subunit 2, cathepsin D light chain, MMP-1, MMP-2, MMP-3, MMP-7, MMP-8, MMP-9, MMP-10, MMP-13, MMP-14, mucin-1, alpha-1-acid glycoprotein, antithrombin, serpin peptidase inhibitor, CTSF, HMGB1, TLR7, COPS2, NT5E, TERF1, TIMP-1, TIMP-2, TIMP-4, XPNPEP2, TGFR2, SIGLEC6, CPE, GHR, GPNMB, SLAMF8, TNFRSF19, TWEAK, IFNGR1, Notch-3, TNFRSF19L, annexin A6, α-defensin-1, caveolin 1, EGF receptor, integrin beta 4, S100A6, S100A8, S100A9, and haptoglobin to create a biomarker profile, and (b) comparing the measured levels of the biomarker profile with reference levels for the biomarkers, wherein a decrease and/or increase in levels of the one or more biomarkers in the biomarker profile relative to the reference levels is indicative of a specific gastroesophageal disease. In a first example of the method, the method further comprises: (c) if, based on the measured levels of the biomarker profile relative to the reference levels the individual is determined to have the specific gastroesophageal disease, administering a treatment to the individual for the specific gastroesophageal disease, wherein the treatment includes one or more of antacids, H-2 receptor blockers, proton pump inhibitors, surgery, transoral incisionless fundoplication, a LINX device, fundoplication, prokinetics, acupuncture, surgical intervention, resection, and chemotherapy. In a second example of the method, optionally including the first example, the decrease and/or increase in levels of the one or more biomarkers in the biomarker profile relative to the reference levels is indicative of one of gastroesophageal reflux disease (GERD), laryngopharyngeal reflux (LPR), reflux laryngitis (RL), nonerosive reflux disease (NERD), and gastric cancer. In a third example of the method, optionally including one or both of the first and second examples, the decrease and/or increase in levels of the one or more biomarkers in the biomarker profile relative to the reference levels is indicative of a specific stage of the specific gastroesophageal disease. In a fourth example of the method, optionally including one or more or each of the first through third examples, the method further comprises: (c) administering a gastrointestinal endoscopy examination to the individual if levels of any two or more salivary biomarkers are within respective threshold ranges, wherein the threshold ranges are E-cadherin ≥5 ng/ml, EGF≥978 pg/mL, TGF-α≥19.2 pg/mL, pepsin ≥92.4 ng/mL, MMP-7≥724 pg/mL, MMP-2≥12.4 pg/mL, MMP-1≥78 pg/ml, MMP-3≥100 ng/ml, MMP-8≥245 ng/mL, MMP-9≥95 ng/ml, MMP-10≥90 pg/ml, MMP-13≥0.05 ng/ml, S100A8≥0.4 pg/ml, S100A9≥91 ng/ml, haptoglobin ≥15 μg/mL, TIMP-1≤291 pg/ml, and/or TIMP-2≤190 pg/ml. In a fifth example of the method, optionally including one or more or each of the first through fourth examples, the method further comprises: (c) administering a gastrointestinal endoscopy examination to the individual if levels of any two or more salivary biomarkers are within respective threshold ranges of respective target values, wherein the target values are E-cadherin 5 ng/ml, EGF 978 pg/mL, TGF-α 19.2 pg/mL, pepsin 92.4 ng/mL, MMP-7 724 pg/mL, MMP-2 12.4 pg/mL, MMP-1 78 pg/ml, MMP-3 100 ng/ml, MMP-8 245 ng/mL, MMP-9 95 ng/ml, MMP-10 90 pg/ml, MMP-13 0.05 ng/ml, S100A8 0.4 pg/ml, S100A9 91 ng/ml, haptoglobin 15 μg/mL, TIMP-1 291 pg/ml, and/or TIMP-2 190 pg/ml, and wherein each threshold range is plus and minus 10% of the respective target value.

The disclosure also provides support for a method of monitoring effectiveness of a treatment for a gastroesophageal disease, comprising: (a) testing a first saliva sample from an individual for levels of one or more biomarkers selected from a group of biomarkers consisting of E-cadherin, TGF-α, EGF, IL-6, pepsin, MOB kinase activator 1a, EphA1, MOB kinase activator 1B, integrin alpha 5, Golgi resident protein GCP 60, FR-beta, protein SEC13 homolog, epiregulin, FGF-12, inhibitor of nuclear factor kappa B kinase catalytic subunit, vimentin, annexin A1, protein S100-A6, malate dehydrogenase, proteasome activator complex subunit 2, cathepsin D light chain, MMP-1, MMP-2, MMP-3, MMP-7, MMP-8, MMP-9, MMP-10, MMP-13, MMP-14, mucin-1, alpha-1-acid glycoprotein, antithrombin, serpin peptidase inhibitor, CTSF, HMGB1, TLR7, COPS2, NT5E, TERF1, TIMP-1, TIMP-2, TIMP-4, XPNPEP2, TGFR2, SIGLEC6, CPE, GHR, GPNMB, SLAMF8, TNFRSF19, TWEAK, IFNGR1, Notch-3, TNFRSF19L, annexin A6, α-defensin-1, caveolin 1, EGF receptor, integrin beta 4, S100A6, S100A8, S100A9, and haptoglobin to create a first biomarker profile, (b) treating the individual for the gastroesophageal disease, (c) taking a second saliva sample from the individual at a time point after treatment for the gastroesophageal disease has begun, (d) testing the second saliva sample for levels of the one or more biomarkers to create a second biomarker profile, and (e) comparing the first biomarker profile and the second biomarker profile, wherein a decrease and/or increase in levels of the one or more biomarkers in the second biomarker profile relative to the first biomarker profile indicates that the treatment is effective. In a first example of the method, the first saliva sample and the second saliva sample are tested using a same type of assay.

References to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

### EXAMPLES

The following studies are provided to illustrate the invention, but are not intended to limit in any way the scope of the invention. Provided herein is the determination of a panel of biomarkers in human whole saliva (WS) and stimulated saliva useful in the prediction, diagnosing and monitoring of gastroesophageal disease. Biomarkers were measured using ELISA and LC-MS tests and biomarkers were evaluated for utility in discriminating amongst gastroesophageal diseases and normal controls.

### Example 1

Salivary Detection of E-Cadherin, TGF-α, EGF, IL-6, MMP-2, MMP-7, and Pepsin in Controls and Those Suffering from Gastroesophageal Disease

Study 1 was conducted to determine the levels of E-cadherin, TGF-α, Epidermal Growth Factor, IL-6, MMP-2 and MMP-7 and pepsin in a reference population. At the time of enrollment, all subjects completed the Reflux Symptom Index (RSI) questionnaire in which a score of greater than 13 was considered abnormal, and answered a detailed questionnaire about their general health, GERD-related symptoms, and their digestive and otorhinolaryngological manifestations (Eckley C A. Estudo da concentragção salivar do fator de crescimento epidérmico em indivíduos com laringite crônica por refluxo laringofaríngeo. São Paulo, 2002; Belafsky P C, Postma G N, and Koufman J A. Validity and reliability of the reflux symptom index (RSI). Journal of Voice. 2002. 16(2): 274-277). They also underwent a videolaryngoscopy exam with a 3.5 mm Pentax flexible scope, which was classified by using the score established by Belafasky, et al. in 2001, in which an RFS score of more or equal to 7 is considered as diagnostic of Laryngopharyngeal Reflux Disease (Reflux Finding Score (RFS) Belafasky P C, Postma G N, Koufman J A. The validity and reliability of the Reflux Finding Score (RFS). Laryngoscope 2001; 111(8):1313-7). Patients with a history of prior esophageal or gastric surgery, or a known esophageal motor disorder were excluded.

The esophageal acid exposure time (AT) and reflux-symptom parameters were used to classify the subjects. All patients with total AT>4.2% were classified as having GERD (n=34). Patients with a normal AT but with a positive symptom association analysis (SAP>95%) were classified as having hypersensitive esophagus (n=30). Patients with a normal AT and a negative symptom association analysis (SAP 20<95%) were classified as having functional heartburn (n=32) (Drossman D A. The functional gastrointestinal disorders and the Rome III process. Gastroenterology 2006; 130:1377-90). A Reflux Disease Questionnaire (RDQ) was administered to each subject. (Shaw M J, Talley N J, Beebe T J, et al. Initial validation of a diagnostic questionnaire for gastroesophageal reflux disease. Am J Gastroenterol 2001; 96:52-25 7).

Reflux monitoring was performed using impedance-pHmetry (MII-pH) (Sandhill Scientific, Colorado, USA). Reflux episodes were determined as described by Sifrim et al. (Sifrim D, Castell D, Dent J, et al. Gastro-oesophageal reflux monitoring: review and consensus report on detection and definitions of acid, non-acid, and gas reflux. Gut 2004; 53:1024-31). Proximal reflux episodes were considered when the refluxate reached the 15 cm impedance sensor. SAP was used to distinguish the association between reflux and symptoms (Weusten B L, Roelofs J M, Akkermans L M, et al. The symptom-association probability: an improved method for symptom analysis of 24-hour esophageal pH data. Gastroenterology 1994; 107:1741-5.).

Saliva samples were taken from each subject. At least 10 minutes prior to collection of unstimulated saliva samples, subjects were asked to rinse orally with water and were asked to relax for 5-15 minutes. They were then seated in a bent forward position in an ordinary chair and asked to put their tongues on the lingual surfaces of the upper incisors and to allow the saliva to drip into sterile plastic (glass) tubes treated with 50 g of 2% sodium azide solution, to prevent microbial decomposition of saliva. The tubes were held to the lower lip for 10 minutes resulting in a collection of 1-5 ml of saliva per individual. Saliva samples were then centrifuged using a Sorvall RT6000D centrifuge (Sorvall, Minn.) at 1800 rpm for 5 minutes to remove debris and were then immediately frozen at −80° C., to await further analysis.

Levels of salivary E-cadherin, TGF-α, EGF, IL-6, MMP-2, MMP-7, and pepsin were measured. Salivary EGF, MMP-2, MMP7, and TGF-α concentrations were measured by using an ELISA kit (Quantikine R, R&D Systems Inc., USA). Salivary pepsin concentrations were measured using the human pepsin ELISA kit (CUSABIO, HuBei, China). IL-6 levels were measured using the commercial kit ELISA (Thermo Fisher Scientific, Waltham, Mass., USA).

Area under the curve (AUC) using a receiver operating characteristic analysis was used to determine the screening ability of salivary biomarkers to predict classification. The area under the receiver operating characteristic curve (AUC) was calculated for determining the prognostic accuracy of the salivary biomarkers. Data were analyzed by using Statistical Package for the Social Sciences (SPSS version 22; IBM Corporation, Armonk, N.Y.).

Table 1 shows the results of the mean and one standard deviation of the levels of the biomarkers for the four study groups/classifications.

TABLE 1
Level of Salivary Biomarkers

| | | | |
|---|---|---|---|
| | | Functional | Hypersensitive |
| Controls | GERD | heartburn | esophagus |
| n = 40 | n = 34 | n = 32 | n = 34 |

Gender

| Male number | 20 | | 15 | 16 | 18 |
|---|---|---|---|---|---|
| Female number | 20 | | 19 | 16 | 16 |
| Mean Age (StdDev) | 45.6 | (4.3) | 43.2 (3.8) | 44.2 (3.2) | 43.6 (2.4) |
| Age Range | 36.4-53.7 | | 38.2-48.9 | 38.2-52.9 | 37.6-48.6 |
| E-cadherin (ng/ml) | 3.6 | (1.2) | 10.8 (1.4) | 2.4 (1.3) | 3.5 (3.2) |
| EGF (pg/mL) | 564.2 | (92.3) | 1783.4 (301.2) | 621.4 (126.8) | 608.5 (108.4) |
| TGF-α (pg/mL) | 10.6 | (1.6) | 24.4 (2.6) | 10.4 (1.8) | 11.2 (1.4) |
| pepsin (ng/mL) | 75.6 | (5.4) | 158.7 (21.3) | 67.8 (13.5) | 54.3 (12.5) |
| MMP-7 (pg/mL) | 567.3 | (62.4) | 1067.5 (102.3) | 652.1 (115.4) | 503.5 (103.6) |
| MMP-2 (pg/mL) | 9.5 | (1.2) | 20.3 (2.3) | 7.6 (1.4) | 6.8 (2.3) |
| RFS mean score (StdDev) | 3.4 | (1.5) | 21.4 (1.3) | 3.5 (1.3) | 3.8 (1.4) |
| RSI mean score (StdDev) | 4.2 | (2.4) | 23.9 (5.7) | 4.5 (1.4) | 4.2 (1.3) |

As shown in Table 1, salivary E-cadherin, TGF-α, EGF, IL-6, MMP-2, MMP-7, and pepsin levels were significantly higher in GERD as compared to functional heartburn, hypersensitive esophagus, and controls (p<0.005). These biomarkers additionally had a significant positive correlation with 24 h AET (r=0.65-0.81, p<0.005), and total number of reflux episodes (r=0.61-0.87, p<0.005) in individuals with GERD.

A statistical comparison of controls and individuals previously diagnosed with GERD and other patients were performed using the two-tailed t-test using GraphPad Prism for Windows, v. 5.01 (GraphPad Software, San Diego, Calif.). Receiver operating characteristic curves (ROC) were generated using the R software environment for statistical computing and graphics (R Foundation for Statistical Computing, Vienna, Austria).

TABLE 2
Area Under the Curve for Distinguishing Between GERD
and Controls Utilizing Combination of Biomarkers

| | Individual | | | | | | |
|---|---|---|---|---|---|---|---|
| | biomarker | TGF-α | EGF | IL-6 | MMP-2 | MMP-7 | Pepsin |
| E-cadherin | 0.88 | 0.92 | 0.93 | 0.92 | 0.93 | 0.91 | 0.92 |
| | (0.82-1.00) | (0.85-1.00) | (0.86-1.00) | (0.82-1.00) | (0.84-1.00) | (0.81-1.00) | (0.81-0.96) |
| TGF-α | 0.79 | | 0.87 | 0.82 | 0.81 | 0.81 | 0.86 |
| | (0.75-0.94) | | (0.82-0.95) | (0.75-0.92) | (0.71-0.84) | (0.73-0.83) | (0.75-1.00) |
| EGF | 0.87 | | | 0.91 | 0.91 | 0.89 | 0.90 |
| | (0.79-1.00) | | | (0.74-0.98) | (0.75-1.00) | (0.74-0.98) | (0.78-1.00) |
| IL-6 | 0.79 | | | | 0.85 | 0.82 | 0.81 |
| | (0.68-0.94) | | | | (0.76-0.98) | (0.73-0.97) | (0.75-0.89) |
| MMP-2 | 0.78 | | | | | 0.85 | 0.81 |
| | (0.74-0.94) | | | | | (0.74-1.00) | (0.68-0.91) |
| MMP-7 | 0.80 | | | | | | 0.84 |
| | (0.69-0.92) | | | | | | (0.76-0.98) |

As shown in Table 2, E-cadherin, TGF-α, EGF, IL-6, MMP-2, MMP-7, and pepsin concentrations were increased in subjects with GERD (AUC 0.79-0.93; p=0.005). Furthermore, the combinations of E-cadherin with EGF, E-cadherin and MMP-2, E-cadherin and pepsin, E-cadherin and MMP 7, E-cadherin and IL-6, EGF and IL-6, EGF and MMP 2, EGF and MMP-2, EGF and MMP-7, and EGF and pepsin showed higher screening, diagnosis, detection and monitoring and prognosis of utility for GERD (p=0.005).

TABLE 3
Correlations of Biomarkers and Age All Subjects

| | EGF | E-cadherin | TGF-α | IL-6 | MMP-2 | MMP-7 | Pepsin |
|---|---|---|---|---|---|---|---|
| R | 0.08 | 0.01 | 0.01 | 0.07 | 0.05 | 0.06 | 0.12 |
| p- | 0.82 | 0.89 | 0.82 | 0.71 | 0.72 | 0.44 | 0.62 |
| Value | | | | | | | |

As shown in Table 3, no correlations were found between age and the level of biomarkers, therefore these biomarkers can be effectively used for the screening, diagnosis, detection, monitoring, or prognosis for GERD, LPR, RL, and NERD for all age groups.

### Example 2

### Analysis of the Diagnostic Performance of Biomarkers in Distinguishing Between Erosive Reflux Disease, Non-Erosive Reflux Disease, and Gastric Cancer

The aim of the study was to distinguish between erosive reflux disease (ERD) and NERD by using salivary E-cadherin, TGF-α, EGF, IL-6, MMP-2, MMP-7, and pepsin, MMP-1, MMP-2, MMP-3, MMP-8, MMPP14, MMP-9, MMP-10, MMP-13, TIMP-1, TIMP-2, S100A8, S100A9, and haptoglobin based kits.

20 ERD, 25 NERD, and 15 gastric cancer patients were selected. The ERD group was selected based on Los Angeles classification i.e., patients with visible esophageal mucosa breaks such as ulcers and/or erosions (Lundell, L. R., Dent, J., Bennett, J. R., Blum, A. L., Armstrong, D., Galmiche, J. P., Johnson, F., Hongo, M., Richter, J. E., Spechler, S. J., Tytgat, G. N., Wallin, L. (1999). Endoscopic assessment of eosophagitis: clinical and functional correlates and further validation of the Los Angeles classification. Gut, 45 (2), 172-180). The NERD group was defined as having symptoms (heartburn and/or acid regurgitation) with known quality and duration appearing at least twice a week for at least six months without any visible esophageal mucosa breaks (Dixon, M. F., Genta, R. M., Yardley, J. H., Correa, P. (1996)). Classification and grading of gastritis. The updated Sydney System. International workshop on the histopathology of gastritis, Houston 1994. Amer. J. Surg. Pathol., 20 (10), 1161-1181). 15 subjects diagnosed with stage 1 gastric cancer were diagnosed based on pathological analysis and according to the Tumor Node Metastasis (TNM) classification. The saliva samples were obtained from each subject as in the same manner as described in Example 1. Salivary biomarkers levels were measured by using ELISA kits as described in Example 1.

Salivary MMP-1 and MMP-3 were analyzed using in-house-developed ELISA (purified MMP-1 and MMP-3 recombinant protein were used as immunogens to generate anti-human MMP-1 and MMP-3 mouse monoclonal antibody, and as 20-protein standards in subsequent ELISA). Salivary S100A8 and S100A9 were analyzed using in-house-developed ELISA (purified S100A8 and S100A9 recombinant protein were used as immunogens to generate anti-human S100A8 and S100A9 mouse monoclonal antibodies, and as protein standards in subsequent ELISA).

MMP-2 levels were determined using human MMP-2 Quantikine ELISA kit (R&D Systems #DMP2F0, USA), MMP-10 levels were determined using human MMP-10-Plex Kit (R&D Systems, USA). MMP-9, MMP-8 and TIMP-1 levels were determined using ELISA kits according to the manufacturer's instructions (R&D Systems, Minneapolis, Minn., USA; Immundiagnostik, Bensheim, Germany, respectively). MMP-13 levels were determined using an MMP13 ELISA Kit (Proteaimmun GmbH, Berlin, Germany), TIMP-2 levels were determined using a TIMP-2 ELISA kit (Quantikine Human Immunoassay, R&D Systems). S100A9 and S100A8 levels were determined using a human CircuLex S100A9 and S100A8 ELISA kit (MBL, Nagova, Japan) and haptoglobin levels were determined using a human haptoglobin ELISA kit (Abcam, Cambridge, USA).

Table 4 provides the results of the biomarker levels for ERD, NERD, and gastric cancer patients. The values expressed are the mean and one standard deviation.

TABLE 4
Level of salivary biomarkers ERD,
NERD, and gastric cancer patients

| | | | | Gastric |
|---|---|---|---|---|
| | ERD | NERD | | cancer |
| | n = 20 | n = 20 | | N = 15 |
| Age (Years) | 45.4 (4.8) | 46.3 | (4.6) | 45.3 (3.1) |
| E-cadherin | 16.7 (1.6) | 13.2 | (1.7) | 40.4 (2.1) |
| (ng/ml) | | | | |
| EGF (pg/mL) | 2308.2 (204.6) | 1563.2 | (201.2) | 2346.5 (108.4) |
| TGF-α (pg/mL) | 28.2 (1.9) | 19.4 | (2.3) | 18.3 (1.4) |
| Pepsin (ng/mL) | 167.8 (16.8) | 121.6 | (23.4) | 231.6 (24.2) |
| MMP-7 (pg/mL) | 1178.3 (96.3) | 986.4 | (95.2) | 2083.4 (124.3) |
| MMP-2 (pg/mL) | 23.6 (3.3) | 18.4 | (3.2) | 35.7 (2.8) |
| MMP-1 (pg/ml) | 102.3 (21.5) | 95.3 | (20.3) | 257.3 (12.1) |
| MMP-3 (ng/ml) | 176.3 (22.6) | 143.4 | (25.4) | 246.8 (27.8) |
| MMP-8 (ng/mL) | 451.4 (21.7) | 308.4 | (34.5) | 657.4 (42.3) |
| MMP-9 (ng/ml) | 189.4 (21.6) | 167.4 | (20.5) | 289.4 (32.7) |
| MMP-10 (pg/ml) | 145.3 (20.7) | 132.3 | (11.3) | 246.7 (34.6) |
| MMP-13 (ng/ml) | 0.23 (0.11) | 0.17 | (0.12) | 1.56 (0.24) |
| TIMP-1 (pg/ml) | 245.3 (23.6) | 252.8 | (36.8) | 114.6 (25.1) |
| TIMP-2 (pg/ml) | 132.3 (14.7) | 156.7 | (21.3) | 108.4 (24.6) |
| S100A8 (pg/ml) | 1.2 (0.5) | 0.8 | (0.4) | 4.4 (1.2) |
| S100A9 (ng/ml) | 124.4 (24.5) | 153.5 | (22.8) | 356.8 (24.9) |
| haptoglobin | 34.2 (7.6) | 32.6 | (19.4) | 54.7 (14.5) |
| (μg/mL) | | | | |

Salivary E-cadherin, TGF-α, EGF, MMP-2, MMP-7, pepsin, MMP-1, MMP-3, MMP-8, MMP-9, MMP-10, MMP-13, TIMP-1, TIMP-2, S100A8, S100A9, and haptoglobin levels were significantly changed in gastric cancer as compared to NERD and/or ERD (p=0.005). Furthermore, salivary TGF-α, EGF, and MMP-8, levels were significantly changed in ERD as compared to NERD (p=0.005).

### Example 3

### Analysis of Diagnostic Performance of E-Cadherin, TGF-α, EGF, IL-6, MMP-2, MMP-7, Pepsin, MMP-1, MMP-2, MMP-3, MMP-8, MMP14, MMP-9, MMP-10, MMP-13, TIMP-1, TIMP-2, S100A8, S100A9, and Haptoglobin

This study was conducted to analyze the accuracy of a combination biomarker panel of salivary E-cadherin, TGF-α, EGF, IL-6, MMP-2, MMP-7, pepsin, MMP-1, MMP-2, MMP-3, MMP-8, MMP-14, MMP-9, MMP-10, MMP-13, TIMP-1, TIMP-2, S100A8, S100A9, and haptoglobin in individuals with GERD in comparison to healthy controls.

A statistical comparison of controls and individuals previously diagnosed with GERD (by the combination of the salivary biomarkers in Examples 1 and 2) was performed using the two-tailed t-test using GraphPad Prism for Windows, v. 5.01 (GraphPad Software, San Diego, Calif.). The diagnostic power of each biomarker alone was investigated as described in Example 1 (Table 2) with univariate ROC analysis on each biomarker in order to obtain its ROC curve, ROC area under the curve (AUC) followed by multivariate ROC analysis on combination of biomarkers panel in order to obtain that panel's ROC curve and AUC. The aim was to find a biomarker panel with the highest ROC AUC.

Receiver operating characteristic curves (ROC) were generated using the R software environment for statistical computing and graphics (R Foundation for Statistical Computing, Vienna, Austria).

Table 5 provides the ROC analysis and diagnostic performance for various salivary biomarker combinations, using E-cadherin (A), TGF-α (B), EGF (C), IL-6 (D), MMP-2 (E), MMP-7 (F), pepsin (G), MMP-1 (H), MMP-2 (I), MMP-3 (J), MMP-8 (K), MMP14 (L), MMP-9 (M), MMP-10 (N), MMP-13 (O), TIMP-1(P), TIMP-2 (Q), S100A8 (R), S100A9 (S), and haptoglobin (T) for the diagnosis of and discrimination between subjects with GERD and control subjects.

TABLE 5
ROC Analysis and Diagnostic Performance for Various Biomarker Combinations

| | A | AB | ABC | ABCD | ABCDE | A-F | A-G | A-H | A-I | A-J |
|---|---|---|---|---|---|---|---|---|---|---|
| AUC | 0.88 | 0.93 | 0.94 | 0.95 | 0.96 | 0.97 | 0.97 | 0.98 | 0.98 | 0.99 |
| Sensitivity | 0.83 | 0.85 | 0.86 | 0.87 | 0.89 | 0.89 | 0.90 | 0.92 | 0.93 | 0.94 |
| Specificity | 0.82 | 0.83 | 0.86 | 0.86 | 0.87 | 0.88 | 0.88 | 0.90 | 0.91 | 0.92 |

| | A-K | A-L | A-M | A-N | A-O | A-P | A-Q | A-R | A-S | A-T |
|---|---|---|---|---|---|---|---|---|---|---|
| AUC | 0.99 | 0.99 | 0.99 | 0.99 | 0.99 | 0.99 | 0.99 | 0.99 | 0.99 | 0.99 |
| Sensitivity | 0.95 | 0.96 | 0.97 | 0.97 | 0.98 | 0.98 | 0.98 | 0.98 | 0.99 | 0.99 |
| Specificity | 0.93 | 0.93 | 0.94 | 0.95 | 0.95 | 0.96 | 0.97 | 0.98 | 0.99 | 0.99 |

TABLE 5A
Reference Value Sensitivity (%), Specificity (%),
Positive Predictive (%), Negative Predictive (%),
and Diagnostic Performance for Various Biomarkers

| | | Sensi- | Speci- | Positive | Negative |
|---|---|---|---|---|---|
| | Reference | tivity | ficity | Predictive | Predictive |
| Biomarkers | Value | (%) | (%) | (%) | (%) |
| E-cadherin | 3.6 | 72 | 72 | 71 | 70 |
| (ng/ml) | | | | | |
| EGF (pg/mL) | 601 | 75 | 74 | 72 | 73 |
| TGF-α (pg/mL) | 10 | 69 | 70 | 70 | 70 |
| Pepsin (ng/mL) | 72 | 72 | 71 | 69 | 72 |
| MMP-7 (pg/mL) | 524 | 71 | 70 | 70 | 69 |
| MMP-2 (pg/mL) | 8.3 | 74 | 72 | 73 | 71 |
| MMP-1 (pg/ml) | 95 | 72 | 70 | 71 | 69 |
| MMP-3 (ng/ml) | 120 | 69 | 72 | 60 | 71 |
| MMP-8 (ng/mL) | 289 | 73 | 71 | 72 | 70 |
| MMP-9 (ng/ml) | 160 | 71 | 73 | 70 | 71 |
| MMP-10 (pg/ml) | 128 | 70 | 74 | 69 | 72 |
| MMP-13 (ng/ml) | 0.15 | 70 | 69 | 69 | 70 |
| TIMP-1 (pg/ml) | 110 | 69 | 72 | 69 | 71 |
| TIMP-2 (pg/ml) | 105 | 71 | 73 | 71 | 72 |
| S100A8 (pg/ml) | 0.5 | 69 | 69 | 68 | 69 |
| S100A9 (ng/ml) | 120 | 70 | 72 | 70 | 70 |
| haptoglobin | 31 | 71 | 70 | 69 | 69 |
| (μg/mL) | | | | | |

The ROC analysis established diagnostic sensitivity and specificity for GERD. The combination models E-cadherin (A), TGF-α (B), EGF (C), IL-6 (D), MMP-2 (E), MMP-7 (F), pepsin (G), MMP-1 (H), MMP-2 (I), MMP-3 (J), MMP-8 (K), MMP-14 (L), MMP-9 (M), MMP-10 (N), MMP-13 (O), TIMP-1 (P), TIMP-2 (Q), S100A8 (R), S100A9 (S), and haptoglobin (T). As shown in Table 5, E-cadherin, TGF-α, EGF, IL-6, MMP-2, MMP-7, and pepsin have high diagnostic values for diagnosis of GERD as compared to other combination models as well as individual biomarkers only. Furthermore, the combination of E-cadherin, EGF, and pepsin and the combination of two biomarkers such as EGF and pepsin have high diagnostic values for diagnosis of GERD as compared to other combination models as well as individual biomarkers only.

### Example 4

### Salivary Biomarkers in Detection of Gastric Cancer

The study included 20 subjects diagnosed with stage 1 gastric cancer based on pathological analysis and according to the Tumor Node Metastasis (TNM) classification. The study additionally included 15 subjects that were recruited as normal healthy subjects. Demographic parameters of the cancer patients and normal healthy subjects are shown in Table 6. There was no significant difference in age or sex of both groups. Saliva samples were collected as described in Example 1. Salivary biomarkers were analyzed or measured by using LC-MS/MS.

TABLE 6
Demographic parameters of both groups

| | Cancer patients | Control subjects |
|---|---|---|
| Number | 20 | 15 |
| F/M | 10/10 | 8/7 |
| Age (years) | 48.5-54.9 | 47.3-55.9 |
| Tumor location | Tunica mucosa | NA |
| Tumor diameter (cm) | 3-5 cm | NA |
| Histological typing | Undifferentiated | |
| Treatment | No | NA |

TABLE 7
Fold change in salivary biomarkers in gastric cancer

| | | Fold | |
|---|---|---|---|
| | Biomarkers | change | p-value |
| 1 | MOB kinase activator 1A | 2.34 | 0.001 |
| 2 | EphA1 | 2.08 | 0.001 |
| 3 | MOB kinase Activator 1B | 1.53 | 0.001 |
| 4 | Integrin alpha 5 | 1.84 | 0.01 |
| 5 | Golgi resident protein GCP 60 | 2.56 | 0.005 |
| 6 | FR-beta | 1.76 | 0.001 |
| 7 | Protein SEC13 homolog | 1.57 | 0.01 |
| 8 | Epiregulin | 2.53 | 0.05 |
| 9 | FGF-12 | 2.16 | 0.01 |
| 10 | Inhibitor of nuclear factor kappa B kinase | 3.68 | 0.01 |
| | catalytic subunit | | |
| 11 | Vimentin | 2.62 | 0.05 |
| 12 | Annexin A1 | 1.87 | 0.01 |
| 13 | Protein S100-A6 | 2.16 | 0.05 |
| 14 | Malate dehydrogenase | 2.89 | 0.001 |
| 15 | Proteasome activator complex subunit 2 | 2.73 | 0.001 |
| 16 | Cathepsin D light chain | 3.86 | 0.005 |
| 17 | MMP-1 | 5.89 | 0.001 |
| 18 | mucin-1 | 4.11 | 0.05 |
| 19 | alpha-1-acid glycoprotein | 3.08 | 0.05 |
| 20 | MMP-2 | 9.86 | 0.001 |
| 21 | Antithrombin | 2.01 | 0.05 |
| 22 | serpin peptidase inhibitor | 1.63 | 0.01 |
| 23 | MMP-3 | 10.54 | 0.001 |
| 24 | CTSF | 3.24 | 0.05 |
| 25 | MMP-8 | 9.76 | 0.001 |
| 26 | HMGB1 | 5.32 | 0.001 |
| 27 | MMP14 | 9.56 | 0.001 |
| 28 | TLR7 | 3.11 | 0.01 |
| 29 | COPS2 | 2.65 | 0.05 |
| 30 | NT5E | 3.99 | 0.05 |
| 31 | MMP-9 | 12.50 | 0.005 |
| 32 | MMP-10 | 11.86 | 0.001 |
| 33 | TERF1 | 4.61 | 0.01 |
| 34 | MMP-13 | 12.06 | 0.001 |
| 35 | TIMP-1 | 9.42 | 0.005 |
| 36 | TIMP-2 | 9.38 | 0.001 |
| 37 | TIMP-4 | 8.53 | 0.001 |
| 38 | XPNPEP2 | 2.07 | 0.01 |
| 39 | TGFR2 | 1.85 | 0.05 |
| 40 | SIGLEC6 | 1.72 | 0.01 |
| 41 | CPE | 1.62 | 0.05 |
| 42 | GHR | 2.54 | 0.01 |
| 43 | GPNMB | 2.50 | 0.05 |
| 44 | SLAMF8 | 1.99 | 0.01 |
| 45 | TNFRSF19 | 1.75 | 0.01 |
| 46 | TWEAK | 1.07 | 0.05 |
| 47 | IFNGR1 | 2.33 | 0.01 |
| 48 | Notch-3 | 4.07 | 0.05 |
| 49 | TNFRSF19L | 3.24 | 0.01 |
| 50 | annexin A6 | 5.63 | 0.005 |
| 51 | α-defensin-1 | 7.84 | 0.001 |
| 52 | caveolin 1 | 1.86 | 0.05 |
| 53 | epidermal growth factor receptor | 14.63 | 0.0001 |
| 54 | integrin beta 4 | 7.64 | 0.005 |
| 55 | S100A6 | 6.52 | 0.01 |
| 56 | S100A8 | 6.09 | 0.001 |
| 57 | S100A9 | 23.82 | 0.0001 |
| 58 | haptoglobin | 10.74 | 0.005 |
| 59 | E-cadherin | 27.06 | 0.0001 |
| 60 | TGF-α | 24.83 | 0.005 |
| 61 | Pepsin | 25.75 | 0.001 |
| 62 | MMP-7 | 21.42 | 0.001 |
| 63 | MMP-2 | 20.74 | 0.005 |

Salivary MOB kinase activator 1A, EphA1, MOB kinase Activator 1B, integrin alpha 5, Golgi resident protein GCP 60, FR-beta, protein SEC13 homolog, epiregulin, FGF-12, inhibitor of nuclear factor kappa B kinase catalytic subunit, vimentin, annexin A1, protein S100-A6, malate dehydrogenase, proteasome activator complex subunit 2, cathepsin D light chain, MMP-1, mucin-1, alpha-1-acid glycoprotein, MMP-2, antithrombin, serpin peptidase inhibitor, MMP-3, CTSF, MMP-8, HMGB1, MMP14, TLR7, COPS2, NT5E, MMP-9, MMP-10, TERF1, MMP-13, TIMP-1, TIMP-2, TIMP-4, XPNPEP2, TGFR2, SIGLEC6, CPE, GHR, GPNMB, SLAMF8, TNFRSF19, TWEAK, IFNGR1, Notch-3, TNFRSF19L, annexin A6, α-defensin-1, caveolin 1, epidermal growth factor receptor, integrin beta 4, S100A6, S100A8, S100A9, haptoglobin, E-cadherin, TGF-α, pepsin, MMP-7, and MMP-2 biomarkers were significantly changed in gastric cancer patients as compared to normal healthy controls.

### Example 5

Analysis of the Use of Biomarkers to Discriminate Between Patients with Gastric Carcinoma, GERD, and Control Patients

Matched gender and aged subjects with gastric carcinoma (diagnosed with stage IA (T1N0M0) gastric cancer, based on biopsy specimen analysis and according to the Tumor Node Metastasis (TNM) classification) and GERD confirmed as well as normal healthy subjects were selected (Table 8). Saliva samples were taken from each subject as described in Example 2. Salivary biomarkers E-cadherin, TGF-α, EGF, IL-6, MMP-2, MMP-7, pepsin, MMP-1, MMP-2, MMP-3, MMP-8, MMPP14, MMP-9, MMP-10, MMP-13, TIMP-1, TIMP-2, S100A8, S100A9, and haptoglobin were measured as described in Example 2.

TABLE 8
Demographic characteristics of the study subjects

| | Gastric | | Normal |
|---|---|---|---|
| | carcinoma | GERD | controls |
| Subjects | 20 | 21 | 20 |
| Gender M/F | 10/10 | 11/10 | 10/10 |
| Age (years) | 64.3 (21.1) | 65.2 (18.5) | 66.3 (19.2) |
| Smokers | 18 | 21 | 20 |

TABLE 9
Level of salivary biomarkers

| | | | | | Gastric | |
|---|---|---|---|---|---|---|
| Biomarker | Controls | | GERD | | carcinoma | |
| E-cadherin | 3.3 | (1.6) | 12.4 | (2.1) | 20.9 | (2.4) |
| (ng/ml) | | | | | | |
| EGF (pg/mL) | 552.2 | (112.3) | 1806.4 | (208.4) | 2234.7 | (214.6) |
| TGF-α (pg/mL | 9.8 | (1.2) | 28.3 | (3.5) | 54.3 | (10.4) |
| pepsin (ng/mL) | 73.5 | (3.7) | 164.3 | (11.3) | 209.7 | (15.3) |
| MMP-7 (pg/mL) | 542.8 | (93.2) | 1006.3 | (125.2) | 1732.4 | (137.8) |
| MMP-2 (pg/mL) | 9.2 | (1.4) | 22.3 | (9.8) | 34.5 | (6.6) |
| MMP-1 (pg/ml) | 61.2 | (12.3) | 106.2 | (18.8) | 260.8 | (27.9) |
| MMP-3 (ng/ml) | 89.3 | (16.5) | 178.3 | (23.6) | 278.9 | (32.5) |
| MMP-8 (ng/mL) | 156.5 | (45.8) | 423.6 | (47.8) | 665.4 | (48.6) |
| MMP-9 (ng/ml) | 87.3 | (24.6) | 178.9 | (24.8) | 291.4 | (36.7) |
| MMP-10 (pg/ml) | 78.4 | (21.4) | 145.5 | (34.8) | 255.4 | (41.2) |
| MMP-13 (ng/ml) | 0.09 | (0.11) | 0.19 | (0.11) | 1.65 | (0.64) |
| TIMP-1 (pg/ml) | 303.6 | (41.8) | 267.4 | (32.6) | 110.6 | (33.7) |
| TIMP-2 (pg/ml) | 247.9 | (32.6) | 178.4 | (43.4) | 107.5 | (23.2) |
| S100A8 (pg/ml) | 0.2 | (0.3) | 0.7 | (0.5) | 4.6 | (1.6) |
| S100A9 (ng/ml) | 95.2 | (22.7) | 164.5 | (33.6) | 323.4 | (25.5) |
| haptoglobin | 11.3 | (2.5) | 24.8 | (12.0) | 61.4 | (13.3) |
| (μg/mL) | | | | | | |

As shown in Table 9, E-cadherin, TGF-α, EGF, IL-6, MMP-2, MMP-7, pepsin, MMP-1, MMP-2, MMP-3, MMP-8, MMP-14, MMP-9, MMP-10, MMP-13, TIMP-1, TIMP-2, S100A8, S100A9, and haptoglobin concentrations were changed in subjects with gastric carcinoma as compared to GERD as well as normal healthy subjects.

### Example 6

### Analysis of the Reproducibility and Stability of Salivary Biomarkers

Saliva samples from 20 patients (10 GERD patients from example 1 and 10 gastric cancer patients from Example 5) and 20 controls (Example 1) were taken from the subjects. The samples were randomly arranged and labeled such that the laboratory could not identify the individuals sampled.

For each analysis, the assay reproducibility of blinded quality control replicates was examined using the coefficient of variation (CV), and a determination was made of the effect of delayed sample processing on analyte concentrations in frozen samples at −80° C. (at twenty four hours, seven days and fourteen days after sampling, i.e. reproducibility with delayed processing). Reproducibility was assessed over a one-week and two-week period for salivary biomarkers, by taking samples at seven days and fourteen days. The CV was determined by estimating the standard deviation (SD) of the quality control values, divided by the mean of these values, multiplied by 100. Inter-observer and intra-observer variances were estimated from repeated sample measurements using a random effects model, with sample identification number as the random variable.

To assess reproducibility, the Intraclass Correlation Coefficient (ICC) values were calculated by dividing the intra-observer variance by the sum of the intra- and inter-observer variances. Ninety-five percent (950%) confidence intervals (CI) were also calculated. The inter- and intra-observer CVs were determined by taking the square root of the inter- and intra-observer variance components from the random effects mixed model on the ln [log] transformed scale, with approximate estimates derived by the eta method. (Rosner B, Fundamentals of Biostatistics. Belmont, Calif.: Duxbury; 2006.) An ICC of <0.40 indicates poor reproducibility, an ICC of 0.40 to 0.8 indicates fair to good reproducibility, and an ICC of more than 0.8 indicates excellent reproducibility. Results are shown in Tables 10 and 11.

Table 10 provides ICCs calculated for delayed analysis and processing of a single frozen sample at day one, day seven, and day fourteen for salivary biomarkers in subjects.

TABLE 10
Intraclass Correlation Coefficient Single Saliva Sample in Subjects

| | | Intra-observer CV | | | Inter-observer CV | | | ICC | | |
|---|---|---|---|---|---|---|---|---|---|---|
| | | (%) | | | (%) | | | (95% CIs) | | |
| | N/time | Day | Day | Day | Day | Day | Day | Day | Day | Day |
| Biomarker | points | 1 | 7 | 14 | 1 | 7 | 14 | 1 | 7 | 14 |
| E-cadherin | 40/3 | 1.4 | 1.2 | 1.5 | 1.9 | 2.1 | 2.5 | 0.91 | 0.91 | 0.91 |
| TGF-α | 40/3 | 1.2 | 1.3 | 1.4 | 2.1 | 2.9 | 3.0 | 0.92 | 0.91 | 0.91 |
| EGF | 40/3 | 1.2 | 1.3 | 1.3 | 2.4 | 2.5 | 2.8 | 0.90 | 0.91 | 0.91 |
| IL-6 | 40/3 | 1.4 | 1.1 | 1.4 | 2.5 | 2.4 | 3.1 | 0.92 | 0.90 | 0.92 |
| MMP-2 | 40/3 | 1.3 | 1.2 | 1.3 | 2.6 | 2.6 | 2.7 | 0.91 | 0.91 | 0.90 |
| MMP-7 | 40/3 | 1.3 | 1.3 | 1.6 | 2.2 | 2.8 | 2.6 | 0.90 | 0.91 | 0.91 |
| pepsin | 40/3 | 1.1 | 1.1 | 1.5 | 2.1 | 2.9 | 2.5 | 0.90 | 0.91 | 0.92 |
| MMP-1 | 40/3 | 1.2 | 1.3 | 1.4 | 2.1 | 2.3 | 2.4 | 0.92 | 0.92 | 0.91 |
| MMP-2 | 40/3 | 1.3 | 1.2 | 1.4 | 2.2 | 2.1 | 2.3 | 0.93 | 0.91 | 0.91 |
| MMP-3 | 40/3 | 1.2 | 1.5 | 1.3 | 1.9 | 2.2 | 2.1 | 0.90 | 0.91 | 0.91 |
| MMP-8 | 40/3 | 1.1 | 1.3 | 1.1 | 2.1 | 2.3 | 2.1 | 0.92 | 0.93 | 0.92 |
| MMP-14 | 40/3 | 1.3 | 1.4 | 1.5 | 2.3 | 2.4 | 2.2 | 0.91 | 0.92 | 0.91 |
| MMP-9 | 40/3 | 1.2 | 1.4 | 1.6 | 2.1 | 2.4 | 2.3 | 0.90 | 0.89 | 0.90 |
| MMP-10 | 40/3 | 1.4 | 1.2 | 1.5 | 2.2 | 2.3 | 2.0 | 0.91 | 0.92 | 0.91 |
| MMP-13 | 40/3 | 1.3 | 1.4 | 1.2 | 2.1 | 2.2 | 2.5 | 0.92 | 0.91 | 0.90 |
| TIMP-1 | 40/3 | 1.5 | 1.6 | 1.4 | 2.2 | 2.1 | 2.4 | 0.91 | 0.90 | 0.92 |
| TIMP-2 | 40/3 | 1.6 | 1.5 | 1.8 | 2.1 | 2.4 | 2.2 | 0.93 | 0.92 | 0.90 |
| S100A8 | 40/3 | 1.2 | 1.3 | 1.4 | 2.2 | 2.5 | 2.3 | 0.90 | 0.91 | 0.90 |
| S100A9 | 40/3 | 1.3 | 1.4 | 1.2 | 2.1 | 2.3 | 2.2 | 0.91 | 0.92 | 0.94 |
| haptoglobin | 40/3 | 1.2 | 1.1 | 1.6 | 2.3 | 2.5 | 2.3 | 0.91 | 0.89 | 0.93 |

Table 11 provide ICCs calculated of samples tested at various time points (day one, day seven and day fourteen) in all subjects.

TABLE 11
Intraclass Correlation Coefficient Time Point Testing in All Subjects

| | | Intra-observer CV | | | Inter-observer CV | | | ICC | | |
|---|---|---|---|---|---|---|---|---|---|---|
| | | (%) | | | (%) | | | (95% CIs) | | |
| | N/time | Day | Day | Day | Day | Day | Day | Day | Day | Day |
| Biomarker | points | 1 | 7 | 14 | 1 | 7 | 14 | 1 | 7 | 14 |
| E-cadherin | 40/3 | 1.3 | 1.2 | 1.4 | 2.3 | 2.3 | 2.7 | 0.92 | 0.90 | 0.91 |
| TGF-α | 40/3 | 1.2 | 1.4 | 1.5 | 2.1 | 2.4 | 2.8 | 0.90 | 0.92 | 0.92 |
| EGF | 40/3 | 1.1 | 1.5 | 1.6 | 2.2 | 2.6 | 2.9 | 0.92 | 0.93 | 0.95 |
| IL-6 | 40/3 | 1.4 | 1.4 | 1.5 | 2.4 | 2.8 | 3.2 | 0.91 | 0.91 | 0.92 |
| MMP-2 | 40/3 | 1.2 | 1.3 | 1.8 | 2.1 | 2.4 | 3.0 | 0.92 | 0.92 | 0.91 |
| MMP-7 | 40/3 | 1.2 | 1.3 | 1.9 | 2.3 | 2.7 | 2.8 | 0.91 | 0.90 | 0.92 |
| pepsin | 40/3 | 1.3 | 1.4 | 1.4 | 2.3 | 2.5 | 2.7 | 0.92 | 0.91 | 0.90 |
| MMP-1 | 40/3 | 1.1 | 1.2 | 1.2 | 2.1 | 2.2 | 2.4 | 0.91 | 0.90 | 0.92 |
| MMP-2 | 40/3 | 1.3 | 1.2 | 1.4 | 2.2 | 2.3 | 2.2 | 0.92 | 0.91 | 0.93 |
| MMP-3 | 40/3 | 1.2 | 1.3 | 1.6 | 2.3 | 2.4 | 2.4 | 0.90 | 0.92 | 0.94 |
| MMP-8 | 40/3 | 1.3 | 1.4 | 1.5 | 2.1 | 2.2 | 2.4 | 0.91 | 0.92 | 0.93 |
| MMP-14 | 40/3 | 1.4 | 1.5 | 1.3 | 2.1 | 2.4 | 2.6 | 0.92 | 0.93 | 0.92 |
| MMP-9 | 40/3 | 1.3 | 1.4 | 1.2 | 2.2 | 2.3 | 2.5 | 0.91 | 0.90 | 0.93 |
| MMP-10 | 40/3 | 1.2 | 1.3 | 1.4 | 2.1 | 2.3 | 2.4 | 0.91 | 0.92 | 0.90 |
| MMP-13 | 40/3 | 1.4 | 1.6 | 1.3 | 2.3 | 2.2 | 2.5 | 0.90 | 0.91 | 0.92 |
| TIMP-1 | 40/3 | 1.3 | 1.4 | 1.2 | 2.2 | 2.4 | 2.5 | 0.91 | 0.93 | 0.89 |
| TIMP-2 | 40/3 | 1.2 | 1.5 | 1.6 | 2.1 | 2.2 | 2.4 | 0.90 | 0.89 | 0.92 |
| S100A8 | 40/3 | 1.3 | 1.4 | 1.6 | 2.1 | 2.4 | 2.6 | 0.90 | 0.89 | 0.89 |
| S100A9 | 40/3 | 1.2 | 1.5 | 1.4 | 2.1 | 2.2 | 2.4 | 0.91 | 0.90 | 0.89 |
| haptoglobin | 40/3 | 1.3 | 1.4 | 1.4 | 2.1 | 2.0 | 2.2 | 0.90 | 0.90 | 0.90 |

The data demonstrate that the ICCs for the range of salivary biomarkers were high (ICCs of 0.9-0.95), indicating good to excellent reproducibility and stability.

### Example 7

### Preparation of Gold Nanoparticle (AuNP)-Antibody Conjugates

Gold nanoparticles (AuNPs, Sigma-Aldrich, St. Louis, Mo., USA) were used for colorimetric label. The gold nanoparticle (AuNP)-antibody conjugate was primed by adding 50 μl of polyclonal antibody or monoclonal antibody (rabbit anti-pepsin, Anti-E-cadherin, anti-TGF-α, anti-EGF, anti-TL-6, anti-MMP-2, anti-MMP-7 antibody, 120 μg/mL in PBS) into 10 mL of AuNP with 40 nm diameter solution with a pH 8, with 0.1M K₂CO₃ in dynamic stirring for 20-100 minutes at room temperature, and then desolated with 4 mL of bovine Serum Albumin (BSA) (600 in PBS). After 15 minutes the AuNP-antibody conjugate was centrifuged at 5000 rpm for 15 minutes and washed with borate buffer with a concentration of 3 mM with pH 7, two to four times. The resulting conjugate was suspended in a 300 borate buffer at 4° C. until integrated. Difference in AuNP size binding with antibody was analyzed by using a UV-Vis spectrophotometer. The colorimetric performance of the AuNP-antibody conjugate was analyzed by ELISA assays.

### Example 8

### Preparation of Lateral Flow Strips

The lateral flow strip includes a sample pad, absorption pad, nitrocellulose membrane, and conjugate pad. The sample and absorption pads or cellulose filter pad or other pads (Whatman Inc. (Florham Park, N.J., USA) were treated with borate buffer with 40 mM and a pH 7 with 0.06% Tween-20 (Sigma-Aldrich, St. Louis, Mo., USA). The conjugation pad was treated with 2% borate buffer with 40 mM with a pH 7 with 15% sucrose and 0.02% Tween-20. The nitrocellulose membrane was treated with PBS with 10 mM with a pH 7. After drying at 37° C. for 45 minutes, at least six functional membrane pads were kept in a desiccator at room temperature to avoid moisture contamination.

The treated functional pads were used to prepare an immune-chromatographic strip. The test line area 6 mm² was created via dispensing 2.0 μL of monoclonal or polyclonal antibody (anti-pepsin, Anti-E-cadherin, anti-TGF-α, anti-Epidermal Growth Factor, anti-IL-6, anti-MMP-2, anti-MMP-7 antibody, anti MMP-25 1, anti-MMP-2, anti-MMP-3, anti-MMP-8, anti-MMPP14, anti-MMP-9, anti-MMP-10, anti-MMP-13, anti-TIMP-1, anti-TIMP-2, anti-S100A8, anti-S100A9 and anti-haptoglobin 240 pg/mL in PBS) in nitrocellulose membrane (0.4 cm×2.5 cm). The control line was created via dispensing 1.5 μL of secondary antibody such as goat, mouse anti-rabbit IgG antibody in 80 μg/mL in PBS and separated from the test line via 1.2 cm. These lines were incubated for 20-90 minutes at 37° C., blocked with 200 μl of BSA for 20 minutes at room temperature, washed with PBS, and then dried for 6 hours at room temperature.

The conjugation pad with an area 0.6 cm×0.6 cm was produced by loading 20 μl of AuNP-antibody conjugate onto the entire pad and drying it for 1 hour at room temperature. The sample and absorption pads had an area 1.2 cm×1.2 cm. The end of each pad was positioned to overlap each other for proper and smooth flow of sample. The analytical performance of lateral flow strips of E-cadherin, TGF-α, EGF, IL-6, MMP-2, MMP-7, pepsin, MMP-1, MMP-2, MMP-3, MMP-8, MMPP14, MMP-9, MMP-10, MMP-13, TIMP-1, TIMP-2, S100A8, S100A9, and haptoglobin was assessed using artificial saliva samples containing different concentrations of pepsin (0.01, 0.5, 1.0, 2.5, 5.0 pg/mL and 0.01, 0.5, 1.0, 2.5, 5.0 15 ng/mL in PBS) as well as other biomarkers such as E-cadherin (0.01, 0.1, 0.5, 1.0, 2.5, 5.0 pg/mL and 0.01, 0.1, 0.5, 1.0, 2.5, 5.0, 15 ng/mL in PBS), TGF-α (0.01, 0.1, 0.5, 1.0, 2.0, 5.0 pg/mL and 0.01, 0.1, 0.5, 1.0, 2.0, 5.0, 15 ng/mL in PBS), EGF (0.01, 0.1, 0.5, 1.0, 2.5, 5.0 pg/mL and 0.01, 0.1, 0.5, 1.0, 2.5, 5.0, 15 ng/mL in PBS), IL-6 (0.01, 0.5, 1.0, 2.5, 5.0 pg/mL and 0.01, 0.5, 1.0, 2.5, 5.0, 15 ng/mL in PBS), MMP-2 (0.01, 0.1, 0.5, 1.0, 2.5, 5.0 pg/mL and 0.01, 0.1, 0.5, 1.0, 2.5, 5.0, 15 ng/mL in PBS), MMP-7 (0.01, 0.1, 0.5, 1.0, 2.5, 5.0 pg/mL and 0.01, 0.1, 0.5, 1.0, 2.5, 5.0, 15 ng/mL in PBS), MMP-1 (0.01, 0.1, 0.5, 1.0, 2.5, 5.0 pg/mL and 0.01, 0.1, 0.5, 1.0, 2.5, 5.0, 10 ng/mL in PBS), MMP-2 (0.01, 0.1, 0.5, 1.0, 2.5, 10 pg/mL and 0.01, 0.1, 0.5, 1.0, 2.5, 5.0, 10 ng/mL in PBS), MMP-3 (0.01, 0.1, 0.5, 1.0, 2.5, 5.0 pg/mL and 0.01, 0.1, 0.5, 1.0, 2.5, 5.0 ng/mL in PBS), MMP-8 (0.01, 0.1, 0.5, 1.0, 2.5, 5.0 pg/mL and 0.01, 0.1, 0.5, 1.0, 2.5, 5.0, 15 ng/mL in PBS), MMP-14 (0.01, 0.1, 0.5, 1.0, 2.5, 5.0, 10.0 pg/mL and 0.01, 0.1, 0.5, 1.0, 2.5, 5.0, 10 ng/mL in PBS), MMP-9 (0.01, 0.1, 0.5, 1.0, 2.5, 10 pg/mL and 0.01, 0.1, 0.5, 1.0, 2.5, 5.0, 10 ng/mL in PBS), MMP-10 (0.01, 0.1, 0.5, 1.0, 2.5, 5.0 pg/mL and 0.01, 0.1, 0.5, 1.0, 2.5, 5.0, 15 ng/mL in PBS), MMP-13 (0.01, 0.1, 0.5, 1.0, 2.5, 10 pg/mL and 0.01, 0.1, 0.5, 1.0, 2.5, 5.0, 10 ng/mL in PBS), TIMP-1 (0.01, 0.1, 0.5, 1.0, 2.5, 5.0 pg/mL and 0.01, 0.1, 0.5, 1.0, 2.5, 5.0, 15 ng/mL in PBS), TIMP-2 (0.01, 0.1, 0.5, 1.0, 2.5, 5.0 pg/mL and 0.01, 0.1, 0.5, 1.0, 2.5, 5.0, 15 ng/mL in PBS), S100A8 (0.01, 0.1, 0.5, 1.0, 2.5, 10 pg/mL and 0.01, 0.1, 0.5, 1.0, 2.5, 5.0, 10 ng/mL in PBS), S100A9 (0.01, 0.1, 0.5, 1.0, 2.5, 5.0 pg/mL and 0.01, 0.1, 0.5, 1.0, 2.5, 5.0, 15 ng/mL in PBS), and haptoglobin (0.01, 0.1, 0.5, 1.0, 2.5, 5.0 pg/mL and 0.01, 0.1, 0.5, 1.0, 2.5, 5.0, 15 ng/mL in PBS) by mixing with 3% BSA, 0.001% Tween-20, and 1.5% methanol. 100 μL of each sample was placed onto the sample pad of the prepared immunochromatographic strip and allowed to flow for 10 minutes. The colorimetric signal generated by the immuno-reaction was captured by using a digital camera or light with wavelength of 350 nm.

### Example 9

Biomarkers Levels in Reflux Esophagitis after Treatment with Rabeprazole

The elevation of specific biomarkers in the saliva is thought to indicate the presence of active gastric reflux. This study was designed to measure the level of the identified biomarkers in saliva of subjects diagnosed with active gastric reflux, before and after medical treatment for that condition.

Thirty (30) subjects diagnosed with Grade A or greater reflux esophagitis by endoscopic examination were included in the study (Los Angeles classification—Lundell L R, Dent J, Bennett J R, et al. Endoscopic assessment of oesophagitis: clinical and functional correlates and further validation of the Los Angeles classification. Gut. 1999; 45:172-180). All underwent endoscopic assessment 4-6 months before participating in the study. Subjects that were currently on any medications of any diseases or disorders, history of gastrointestinal resection, cancer, and cancer treatment were excluded. All subjects that were enrolled in the study after approval by the local ethical Institutional Review Board and provided written informed consent.

TABLE 12
Subject Characteristics

| | |
|---|---|
| Number of Subjects | 30 |

Gender

| | | |
|---|---|---|
| Male | 16 | (53.3%) |
| Female | 14 | (46.7%) |
| Mean Age (STdDev) | 33.4 | (5.4) |
| Current Smoker | 5 | (16.7%) |

Alcohol use

| | | |
|---|---|---|
| None | 20 | (66.7%) |
| Occasional | 5 | (16.7%) |
| Habitual | 5 | (16.7%) |

Helicobacter pylori testing

| | | |
|---|---|---|
| Negative | 20 | (66.7%) |
| Negative after eradication | 7 | (23.3%) |
| Positive | 3 | (10.0%) |

Once enrolled, subjects received rabeprazole, 10 mg once a day for 6 weeks. Saliva samples were taken before starting treatment and at the end of the six weeks and the concentration of salivary biomarkers (E-cadherin, EGF, TGF-α, pepsin, MMP-7, MMP-2, MMP-1, MMP-3, MMP-8, MMP-9, MMP-10, MMP-13, TIMP-1, TIMP-2, S100A8, S100A9, and haptoglobin) were measured as described in Example 2. The mean and standard deviation for each biomarker at the two time points are shown in Table 13.

TABLE 13
Level of salivary biomarkers

| Biomarker | Enrollment | | 6 weeks | | Controls | |
|---|---|---|---|---|---|---|
| E-cadherin (ng/ml) | 9.4 | (2.3) | 5.2 | (1.4) | 3.3 | (1.6) |
| EGF (pg/mL) | 1356.4 | (235.5) | 523.8 | (167.8) | 552.2 | (112.3) |
| TGF-α (pg/mL) | 22.7 | (2.3) | 14.4 | (3.6) | 9.8 | (1.2) |
| pepsin (ng/mL) | 153.8 | (26.5) | 48.6 | (22.4) | 73.5 | (3.7) |
| MMP-7 (pg/mL) | 1085.3 | (258.3) | 503.3 | (215.6) | 542.8 | (93.2) |
| MMP-2 (pg/mL) | 18.4 | (2.5) | 6.4 | (3.2) | 9.2 | (1.4) |
| MMP-1 (pg/ml) | 268.4 | (13.4) | 198.3 | (3.1) | 61.2 | (12.3) |
| MMP-3 (ng/ml) | 168.4 | (15.7) | 114.8 | (11.3) | 89.3 | (16.5) |
| MMP-8 (ng/mL) | 185.3 | (32.6) | 308.4 | (34.7) | 156.5 | (45.8) |
| MMP-9 (ng/ml) | 198.4 | (27.9) | 98.3 | (15.3) | 87.3 | (24.6) |
| MMP-10 (pg/ml) | 146.7 | (36.4) | 98.2 | (14.2) | 78.4 | (21.4) |
| MMP-13 (ng/ml) | 0.45 | (0.21) | 0.42 | (0.42) | 0.09 | (0.11) |
| TIMP-1 (pg/ml) | 225.2 | (32.6) | 309.2 | (39.7) | 303.6 | (41.8) |
| TIMP-2 (Pg/ml) | 134.5 | (23.3) | 189.5 | (25.5) | 247.9 | (32.6) |
| S100A8 (pg/ml) | 1.7 | (0.5) | 0.6 | (0.3) | 0.2 | (0.3) |
| S100A9 (ng/ml) | 178.5 | (25.6) | 109.4 | (34.4) | 95.2 | (22.7) |
| haptoglobin (μg/mL) | 34.7 | (9.4) | 21.6 | (8.7) | 11.3 | (2.5) |

As seen in Table 13, the level of each of the salivary biomarkers were significantly changed (p<0.001) when comparing the levels at the time of enrollment and 6 weeks after treatment with rabeprazole, with some biomarkers reaching normal levels as shown in comparison to the controls of Example 1.

### Example 10

### Fe—N—C Single-Atom Nanozymes (SANs) Based Kit for Detection of Gastric Cancer

Methyl Orange (MeO) (100 mg) was mixed in distilled water; followed by 1 g of FeCl₃ and 0.3 ml pyrrole were added under v stirring to form a Fe3+-doped Fe-doped polypyrrole nanotube. MnO₂-coated polypyrrole nanotubes were prepared by dispersing 80 uL of KMnO₄ into the aforesaid solution. The product was pyrolyzed at 740° C. under the nitrogen atmosphere, and the MnO₂ coating was washed out by leaching for 7.2 h with 5% H₂SO₄. Fe—N—C single-atom nanozymes were formed after the heat treatment at 600° C. with ammonia.

Fe—N—C single-atom nanozymes were crushed under vigorous sonication and dispersed in PBS with 0.1 mg/ml, followed by pH adjustment with K₂CO₃ and broken down to nanosize via ultrasonication for 45 minutes. The solution was then modulated by N₃-dimethylaminopropyl-N-ethylcarbodiimide with 0.5 mg/ml and N-hydroxysuccinimide with 1 mg/ml stirring for 20 minutes, followed by centrifuging of the mixture and then the mixture was washed four times to form the activated Fe—Nx SANs. A streptavidin-functionalized nanoparticle-enriched carbon nanotube is designed as a trace tag for ultrasensitive multiplexed or single biomarkers measurements of biomarkers. Streptavidin (100 μg/ml in PBS) was incubated with activated Fe—N—C single-atom nanozymes at 32° C. up to 45 minutes, and finally was centrifuged for four times to remove unbonded Streptavidin. Finally, the products were treated with 1% BSA for 15 minutes and dispersed in 0.5 mL of PBS. In this, the Streptavidin functionalized-labeled Fe—N—C single-atom nanozymes were crashed down to nanosize by using an intense ultrasound treatment for 45 minutes. A standard sandwich-based immunoassay was then performed, where each biomarker was detected using corresponding antibodies functionalized with biotin. The resulting chromogenic reaction was measured using, for example, a spectrophotometer.

TABLE 14
Lower detection limit of kit of biomarkers

| | Limit of |
|---|---|
| | detection |
| E-cadherin (pg/ml) | 0.001 |
| EGF (pg/mL) | 0.001 |
| TGF-α (pg/mL) | 0.001 |
| pepsin (pg/mL) | 0.005 |
| MMP-7 (pg/mL) | 0.001 |
| MMP-2 (pg/mL) | 0.001 |
| MMP-1 (pg/ml) | 0.003 |
| MMP-3 (pg/ml) | 0.002 |
| MMP-8 (pg/mL) | 0.001 |
| MMP-9 (pg/ml) | 0.004 |
| MMP-10 (pg/ml) | 0.001 |
| MMP-13 (pg/ml) | 0.002 |
| TIMP-1 (pg/ml) | 0.001 |
| TIMP-2 (Pg/ml) | 0.002 |
| S100A8 (pg/ml) | 0.003 |
| S100A9 (pg/ml) | 0.001 |
| haptoglobin (pg/mL) | 0.001 |

This novel designed single atom nanozymes demonstrates the ultralow limit of detection (LOD) of 0.001 pg/mL of biomarkers levels.

### Example 11

### Antibody Micropatterned Lubricant Infused Kit for Detection of Biomarkers

This kit is designed based on the concept of lubricant-infused surfaces. This provides tunable bioactivity with omniphobic properties to bind the antibodies via biofunctional domains into a lubricant-infused layer. The surface is prepared by mixing self-assembled monolayers of aminosilanes and fluorosilanes. Aminosilanes were utilized as coupling molecules for immobilizing capture ligands, and proteins, i.e., different biomarkers, were prevented from infiltrating the fluorosilane molecules with a thin layer of a biocompatible fluorocarbon-based lubricant.

The process began with washing a P type boron doped silicon wafer with a prepared solution (mixing 60% sulfuric acid and 40% hydrogen peroxide). Then, a negative photoresist DNR-L300-40 was spin coated at 3500 rpm for 60 s followed by soft baking at 90° C. for 120 s on a hot plate. Exposure with a chrome mask was performed for 10 s on a mask aligner. Following that, the wafer was baked at 100° C. for 120 s on a hot plate. The patterns were created by using AZ 300 MIF for 60 s 10 followed by hard baking at 90° C. for 120 s. Titanium 200 Å and gold 1000 Å were deposited on the patterned wafer by using an E-beam evaporator. The wafer was sonicated in acetone for 6 min followed by methanol and isopropyl alcohol wash for 180 s each and followed by rinsing the wafer with deionized water. The gold grid pattern-based chip was soaked for 45 min in the 60% sulfuric acid and 40% hydrogen peroxide solution and then washed with normal saline water. Washing was performed with 1% SDS solution and 1% potassium hydroxide by using an ultrasonic cleaner. After washing with distilled water, the chip was dried with nitrogen gas. To form a protein layer on the gold grid patterns, 0.1 mg/mL proteins such as E-cadherin, TGF-α, EGF, IL-6, MMP-2, MMP-7, pepsin, MMP-1, MMP-2, MMP-3, MMP-8, MMPP14, MMP-9, MMP-10, 5 MMP-13, TIMP-1, TIMP-2, S100A8, S100A9 and haptoglobin were reacted with the chip for 90 mins. These patterns were washed 5 times and 3 min each with 1× phosphate-buffered saline with 0.1% Tween detergent. After washing, the corresponding antibody (e.g., if the protein layer is formed from E-cadherin, the antibody may be anti-E-cadherin) dissolved in PBS and 1 M magnesium chloride was added onto the gold surface and allowed to react for 90 minutes. A standard sandwich-based immunoassay was then performed, using functionalized antibodies and a suitable detection method such as fluorescence.

TABLE 15
Lower detection limit of kit of biomarkers

| | Limit of |
|---|---|
| | detection |
| E-cadherin (pg/ml) | 0.03 |
| EGF (pg/mL) | 0.02 |
| TGF-a (pg/mL) | 0.04 |
| pepsin (pg/mL) | 0.05 |
| MMP-7 (pg/mL) | 0.04 |
| MMP-2 (pg/mL) | 0.02 |
| MMP-1 (pg/ml) | 0.02 |
| MMP-3 (pg/ml) | 0.01 |
| MMP-8 (pg/mL) | 0.03 |
| MMP-9 (pg/ml) | 0.05 |
| MMP-10 (pg/ml) | 0.02 |
| MMP-13 (pg/ml) | 0.02 |
| TIMP-1 (pg/ml) | 0.03 |
| TIMP-2 (Pg/ml) | 0.02 |
| S100A8 (pg/ml) | 0.04 |
| S100A9 (pg/ml) | 0.05 |
| haptoglobin (pg/mL) | 0.06 |

Thus, using the antibody micropatterned lubricant infused kit, biomarkers were detected at very low levels (e.g., 0.02 pg/mL).

### Example 12

### Validation of Diagnosing ERD and NERD by Using Salivary E-Cadherin, TGF-α, EGF, TL-6, MMP-2, MMP-7, Pepsin, MMP-1, MMP-2, MMP-3, MMP-8, MMP14, MMP-9, MMP-10, 5 MMP-13, TIMP-1, TIMP-2, S100A8, S100A9 and Haptoglobin.

50 subjects were selected. Salivary samples were taken from the 50 subjects. Salivary E-cadherin, TGF-α, EGF, IL-6, MMP-2, MMP-7, pepsin, MMP-1, MMP-2, MMP-3, MMP-8, MMP-14, MMP-9, MMP-10, MMP-13, TIMP-1, TIMP-2, S100A8, S100A9, and haptoglobin were measured as described in Example 2. Different biomarker levels in GERD mentioned in Table 5A of Example 3 were taken as cutoff value of biomarkers. Final diagnosis was confirmed by using impedance-pHmetry (MII-pH. Reflux monitoring was performed using impedance-pHmetry (MII-pH) (Sandhill Scientific, Colorado, USA)).

Salivary biomarkers of E-cadherin, TGF-α, EGF, IL-6, MMP-2, MMP-7, pepsin, MMP-15 1, MMP-2, MMP-3, MMP-8, MMP-14, MMP-9, MMP-10, MMP-13, TIMP-1, TIMP-2, S100A8, S100A9 and haptoglobin indicated that 30 subjects were GERD. Further, the 30 subjects whose biomarkers indicated a diagnosis of GERD were confirmed by using impedance-pHmetry, which showed that 29 of the 30 subjects have GERD.

TABLE 16
Biomarker levels in GERD subjects

| | GERD | |
|---|---|---|
| | n = 30 | |
| Age (Years) | 43.4 | (4.3) |
| E-cadherin (ng/ml) | 14.3 | (2.1) |
| EGF (pg/mL) | 2219 | (105.8) |
| TGF-α (pg/mL) | 24.4 | (3.4) |
| Pepsin (ng/mL) | 145.7 | (21.3) |
| MMP-7 (pg/mL) | 1056.4 | (57.8) |
| MMP-2 (pg/mL) | 22.4 | (3.1) |
| MMP-1 (pg/ml) | 101.6 | (19.6) |
| MMP-3 (ng/ml) | 157.3 | (16.7) |
| MMP-8 (ng/mL) | 432.6 | (20.5) |
| MMP-9 (ng/ml) | 178.9 | (15.6) |
| MMP-10 (pg/ml) | 142.2 | (18.6) |
| MMP-13 (ng/ml) | 0.20 | (0.1) |
| TIMP-1 (pg/ml) | 247.6 | (34.3) |
| TIMP-2 (pg/ml) | 145.7 | (20.3) |
| S100A8 (pg/ml) | 1.1 | (0.3) |
| S100A9 (ng/ml) | 149.4 | (21.5) |
| haptoglobin(μg/mL) | 33.5 | (7.3) |

Thus, salivary biomarkers of E-cadherin, TGF-α, EGF, IL-6, MMP-2, MMP-7, pepsin, MMP-1, MMP-2, MMP-3, MMP-8, MMP-14, MMP-9, MMP-10, MMP-13, TIMP-1, TIMP-2, S100A8, S100A9, and haptoglobin are highly predictive for diagnosis and the effectiveness of treatment of GERD.

### Example 13

Salivary Detection and Validation of EGF and Pepsin in Controls and Those Suffering from GERD

This study was conducted to determine the levels of EGF and pepsin in a reference population. At the time of enrollment, all the subjects completed the Reflux Symptom Index (RSI) questionnaire, and answered a detailed questionnaire about their general health, GERD-related symptoms, and their digestive and otorhinolaryngological manifestations as described above in Example 1. Patients with a history of prior esophageal or gastric surgery, or a known esophageal motor disorder were excluded. Saliva samples were taken from each subject as described in Example 1. Levels of salivary EGF and pepsin were measured as described in Example 1. Data were analyzed by using Statistical Package for the Social Sciences (SPSS version 22; IBM Corporation, Armonk, N.Y.). A statistical comparison of controls and individuals previously diagnosed with GERD (by the combination of the salivary biomarkers such as EGF and pepsin) was performed using the two-tailed t-test using GraphPad Prism for Windows, v. 5.01 (GraphPad Software, San Diego, Calif.). Univariate ROC analysis on each biomarker was performed in order to obtain ROC curves and ROC area under the curve (AUC) followed by multivariate ROC analysis on the combination of biomarkers in order to obtain that combination's ROC curve and AUC. Table 17 shows the results (mean and one standard deviation) of the levels of the biomarkers for the four study groups/classifications.

TABLE 17
Level of Salivary Biomarkers

| | |
|---|---|
| Controls | GERD |
| n = 25 | n = 49 |

Gender

| | | |
|---|---|---|
| Male (number) | 17 | 18 |
| Female (number) | 8 | 31 |
| Mean Age (StdDev) | 46.6 (14.2) | 49.2 (16.7) |
| EGF (ng/mL) | 8.2 (1.8) | 13.3 (2.9) |
| pepsin (ng/mL) | 32.1 (10.3) | 128.3 (10.8) |
| RSI mean score (StdDev) | 3.5 (1.4) | 22.8 (3.5) |

TABLE 18
Reference Value Sensitivity (%), Specificity (%),
Positive Predictive (%), Negative Predictive (%),
and Diagnostic Performance for Various Biomarkers

| | | Sensi- | Speci- | Positive | Negative |
|---|---|---|---|---|---|
| | Reference | tivity | ficity | Predictive | Predictive |
| Biomarkers | Value | (%) | (%) | (%) | (%) |
| EGF (ng/mL) | 9.0 | 73 | 72 | 71 | 72 |
| Pepsin (ng/mL) | 42 | 100 | 98 | 98 | 98 |
| Pepsin and EGF | Pepsin 42 | 100 | 100 | 100 | 100 |
| | EGF 9.0 | | | | |

As shown in Table 17, salivary EGF and pepsin levels were significantly higher in GERD as compared to and controls (p<0.005). The combination of salivary EGF and pepsin leads to higher diagnostic value as compared to either single biomarker (as shown in Table 18).

### Example 14

Analysis of the Use of Biomarkers to Differentiate Between Patients with Different Staging of Gastric Cancer, Different Types of GERD Such as Mild, Moderate, and Severe.

Matched gender and aged subjects with gastric carcinoma (diagnosed with stage IA (T1N0M0), IIA, IIIA gastric cancer, based on biopsy specimen analysis and according to the Tumor Node Metastasis (TNM) classification) and GERD of different staging (mild, moderate, and severe) confirmed as shown in table 19 (Chandrasoma, P. New evidence defining the pathology and pathogenesis of lower esophageal sphincter damage. Eur Surg 51, 282-290 (2019)) were selected (Table 8). Saliva samples were taken from each subject as described in Example 2. Salivary biomarkers E-cadherin, TGF-α, EGF, IL-6, MMP-2, MMP-7, pepsin, MMP-1, MMP-2, MMP-3, MMP-8, MMP14, MMP-9, MMP-10, MMP-13, TIMP-1, TIMP-2, S100A8, S100A9, and haptoglobin were measured as described in Example 2.

TABLE 19
Demographic characteristics of the study subjects

| | Gastric carcinoma | | | GERD | | |
|---|---|---|---|---|---|---|
| | IA | IIA | IIIA | Mild | Moderate | Severe |
| Subjects | 10 | 10 | 12 | 12 | 10 | 10 |
| Gender M/F | 5/5 | 5/5 | 7/5 | 6/6 | 5/5 | 5/5 |
| Age (years) | 54.2 | 53.5 | 54.4 | 53.8 | 52.6 | 54.6 |
| | (12.3) | (4.2) | (3.1) | (2.6) | (2.9) | (1.8) |

TABLE 20
Level of salivary biomarkers

| | GERD | | | Gastric carcinoma | | |
|---|---|---|---|---|---|---|
| Biomarker | Mild | Moderate | Severe | IA | IIA | IIIA |
| E-cadherin | 9.8 | 12.5 | 15.4 | 19.7 | 23.6 | 27.9 |
| (ng/ml) | (1.5) | (1.4) | (1.1) | (2.1) | (3.3) | (2.6) |
| EGF | 1509.4 | 1685.2 | 1874.3 | 2156.5 | 2362.4 | 2543.6 |
| (pg/mL) | (113.4) | (209.6) | (287.5) | (256.3) | (301.2) | (287.6) |
| TGF-α | 18.9 | 23.6 | 31.6 | 42.5 | 53.6 | 56.9 |
| (pg/mL) | (3.4) | (2.4) | (3.6) | (4.5) | (4.7) | (7.4) |
| pepsin | 134.6 | 165.3 | 180.5 | 198.4 | 234.5 | 278.8 |
| (ng/mL) | (12.9) | (13.1) | (14.2) | (14.3) | (13.2) | (14.6) |
| MMP-7 | 987.6 | 1156.7 | 1261.9 | 1462.6 | 1674.9 | 1894.3 |
| (pg/mL) | (125.4) | (139.3) | (128.4) | (148.9) | (158.3) | (154.6) |
| MMP-2 | 18.6 | 21.3 | 25.4 | 31.7 | 38.5 | 45.8 |
| (pg/mL) | (5.6) | (2.9) | (4.4) | (5.1) | (4.6) | (5.3) |
| MMP-1 | 100.8 | 134.6 | 149.6 | 198.5 | 245.9 | 308.4 |
| (pg/ml) | (19.3) | (14.3) | (18.5) | (119) | (14.6) | (23.1) |
| MMP-3 | 144.5 | 171.3 | 201.2 | 225.9 | 267.3 | 308.4 |
| (ng/ml) | (15.6) | (20.3) | (19.3) | (14.5) | (21.1) | (16.3) |
| MMP-8 | 387.5 | 434.4 | 507.8 | 567.2 | 652.3 | 791.4 |
| (ng/mL) | (32.7) | (41.7) | (42.6) | (45.9) | (52.3) | (47.5) |
| MMP-9 | 150.8 | 177.1 | 209.5 | 256.7 | 289.6 | 350.4 |
| (ng/ml) | (25.6) | (24.3) | (21.6) | (24.2) | (34.2) | (31.2) |
| MMP-10 | 109.4 | 123.4 | 194.9 | 250.3 | 325.8 | 403.7 |
| (pg/ml) | (42.1) | (39.7) | (41.7) | (33.5) | (31.9) | (45.2) |
| MMP-13 | 0.14 | 0.21 | 0.87 | 1.09 | 1.63 | 2.78 |
| (ng/ml) | (0.08) | (0.09) | (0.21) | (0.32) | (0.38) | (0.42) |
| TIMP-1 | 253.7 | 201.5 | 158.3 | 107.8 | 87.1 | 45.6 |
| (pg/ml) | (30.8) | (27.6) | (20.3) | (22.7) | (20.4) | (28.9) |
| TIMP-2 | 207.2 | 189.4 | 121.7 | 98.5 | 76.9 | 52.8 |
| (pg/ml) | (39.3) | (33.5) | (35.9) | (32.5) | (28.5) | (31.6) |
| S100A8 | 0.5 | 1.2 | 2.3 | 3.9 | 4.8 | 6.8 |
| (pg/ml) | (0.3) | (0.4) | (0.5) | (0.6) | (0.5) | (1.3) |
| S100A9 | 158.3 | 209.3 | 249.5 | 304.7 | 354.8 | 409.3 |
| (ng/ml) | (31.4) | (33.6) | (31.4) | (35.6) | (29.4) | (41.7) |
| haptoglobin | 20.6 | 37.7 | 49.3 | 60.3 | 79.7 | 98.8 |
| (μg/mL) | (4.2) | (5.8) | (4.2) | (5.7) | (6.6) | (7.9) |

As shown in Table 20, E-cadherin, TGF-α, EGF, IL-6, MMP-2, MMP-7, pepsin, MMP-1, MMP-2, MMP-3, MMP-8, MMP-14, MMP-9, MMP-10, MMP-13, TIMP-1, TIMP-2, S100A8, S100A9, and haptoglobin concentrations changed in subjects with different stages of gastric carcinoma as compared to different stages of GERD. Furthermore, significant changes in salivary biomarkers were noted in higher stages (i.e., advanced staging) of cancer (i.e., IIIA as compared to IIA and IA). Moreover, significant changes in salivary biomarkers were noted in higher stages of GERD (i.e., advanced staging and severe staging as compared to moderate and mild stage). Thus E-cadherin, TGF-α, EGF, IL-6, MMP-2, MMP-7, pepsin, MMP-1, MMP-2, MMP-3, MMP-8, MMP-14, MMP-9, MMP-10, MMP-13, TIMP-1, TIMP-2, S100A8, S100A9 may also be used for diagnostic, prognostic, treatment, and monitoring of different stages of GERD and cancer.

### Example 15

### The Role of Salivary Biomarkers to Select for Gastrointestinal Endoscopy Examination

A total of 31 subjects were randomly selected and all subjects underwent a 24-hour multichannel intraluminal impedance pH examination, esophageal high-resolution manometry monitoring, upper gastrointestinal imaging, and endoscopy. During the endoscopy and 24-hour multichannel intraluminal impedance pH examination, the subjects were considered as positive for GERD if they had reflux esophagitis, abnormal pH results, or abnormal impedance results, otherwise the subjects were considered negative for GERD. Saliva samples were taken from each subject as described in Example 2. Salivary biomarker levels were measured as described in Example 2. E-cadherin, TGF-α, EGF, IL-6, MMP-2, MMP-7, pepsin, MMP-1, MMP-2, MMP-3, MMP-8, MMPP14, MMP-9, MMP-10, MMP-13, TIMP-1, TIMP-2, S100A8, and S100A9 were correlated with endoscopy examination results. Data were analyzed by using Statistical Package for the Social Sciences (SPSS version 22; IBM Corporation, Armonk, N.Y.).

TABLE 21
Salivary biomarkers in positive and negative endoscopy

| | |
|---|---|
| Negative | Positive |
| endoscopy | endoscopy |
| n = 13 | N = 18 |

| | | | | |
|---|---|---|---|---|
| E-cadherin (ng/ml) | 3.4 | (1.2) | 7.8 | (2.3) |
| EGF (pg/mL) | 524.4 | (109.3) | 1367.5 | (189.4) |
| TGF-α (pg/mL | 8.5 | (1.4) | 25.2 | (3.6) |
| pepsin (ng/mL) | 71.3 | (10.3) | 156.4 | (13.8) |
| MMP-7 (pg/mL) | 542.8 | (93.2) | 1006.3 | (125.2) |
| MMP-2 (pg/mL) | 8.2 | (2.3) | 20.3 | (5.4) |
| MMP-1 (pg/ml) | 54.4 | (12.3) | 98.3 | (10.4) |
| MMP-3 (ng/ml) | 78.4 | (14.2) | 156.4 | (23.1) |
| MMP-8 (ng/mL) | 147.4 | (34.2) | 346.3 | (38.5) |
| MMP-9 (ng/ml) | 78.4 | (22.5) | 153.2 | (34.6) |
| MMP-10 (pg/ml) | 73.3 | (21.4) | 134.3 | (31.4) |
| MMP-13 (ng/ml) | 0.09 | (0.09) | 0.18 | (0.09) |
| TIMP-1 (pg/ml) | 321.6 | (32.5) | 245.4 | (35.6) |
| TIMP-2 (pg/ml) | 256.3 | (42.1) | 156.5 | (32.7) |
| S100A8 (pg/ml) | 0.3 | (0.2) | 0.8 | (0.3) |
| S100A9 (ng/ml) | 84.7 | (24.2) | 153.3 | (23.4) |
| haptoglobin (μg/mL) | 11.4 | (4.5) | 22.3 | (5.2) |

The levels of E-cadherin, TGF-α, EGF, IL-6, MMP-2, MMP-7, pepsin, MMP-1, MMP-2, MMP-3, MMP-8, MMP14, MMP-9, MMP-10, MMP-13, TIMP-1, TIMP-2, S100A8, and S100A9 in saliva were significantly changed in subjects who exhibited a positive endoscopy relative to subjects who exhibited a negative endoscopy (see Table 21). Thus, these biomarkers may be used to make decisions as to whether an endoscopy examination should be performed.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A kit for differentiating among gastroesophageal diseases, the gastroesophageal diseases selected from erosive reflux disease, non-erosive reflux disease, and gastric cancer, the kit comprising:
a solid support on which a plurality of agents have been affixed which in combination bind to salivary EGF, salivary pepsin and one or more additional salivary biomarkers selected from a group of biomarkers consisting of E-cadherin, TGF-α, IL-6, MOB kinase activator 1A, EphA1, MOB kinase Activator 1B, integrin alpha 5, Golgi resident protein GCP 60, FR-beta, protein SEC13 homolog, epiregulin, FGF-12, inhibitor of nuclear factor kappa B kinase catalytic subunit, vimentin, annexin A1, protein S100-A6, malate dehydrogenase, proteasome activator complex subunit 2, cathepsin D light chain, MMP-1, MMP-2, MMP-3, MMP-7, MMP-8, MMP-9, MMP-10, MMP-13, MMP-14, mucin-1, alpha-1-acid glycoprotein, antithrombin, serpin peptidase inhibitor, CTSF, HMGB1, TLR7, COPS2, NT5E, TERF1, TIMP-1, TIMP-2, TIMP-4, XPNPEP2, TGFR2, SIGLEC6, CPE, GHR, GPNMB, SLAMF8, TNFRSF19, TWEAK, IFNGR1, Notch-3, TNFRSF19L, annexin A6, α-defensin-1, caveolin 1, EGF receptor, integrin beta 4, S100A6, S100A8, S100A9, and haptoglobin, wherein each agent binds to a different single biomarker.

2. (canceled)

3. The kit of claim 1, wherein the plurality of agents in combination bind to E-cadherin, salivary EGF, salivary pepsin, and one or more of TGF-α, IL-6, MMP-2, and MMP-7.

4. The kit of claim 1, wherein the plurality of agents in combination bind to salivary EGF, IL-6, MMP-2, MMP-7, salivary pepsin, and MMP-8.

5. The kit of claim 1, wherein the plurality of agents in combination bind to salivary EGF, salivary pepsin, and E-cadherin.

6. The kit of claim 1, wherein the solid support includes a plurality of test strips, each configured to produce a detectable output at a level proportional to a level present on the test strip of salivary EGF, pepsin, and the one or more additional salivary biomarkers after the test strips are exposed to a saliva sample, and wherein when the detectable output levels indicate that salivary EGF, pepsin and/or the one or more of the salivary biomarkers meet one or more criteria in a group of criteria, a gastroesophageal disease of the gastroesophageal diseases is indicated.

7-8. (canceled)

9. The kit of claim 1, wherein the plurality of agents comprise one or more of antibodies, antigens, nanoparticles, aptamers, inhibitors, substrates, cofactors, coenzymes, lectins, nucleic acids, protein A, protein G, nonbiological ligands, boronates, triazine dyes, and metal-ion chelates.

10. (canceled)

11. The kit of claim 1, wherein the kit is an antibody micropatterned lubricant infused kit comprising self-assembled monolayers of aminosilanes.

12. The kit of claim 1, wherein the kit is an Fe—N—C single-atom nanozymes (SANs) based kit comprising streptavidin-functionalized nanoparticle-enriched carbon nanotubes.

13. A method for differentiating between specific gastroesophageal diseases, wherein specific gastroesophageal diseases are selected from erosive reflux disease, non-erosive reflux disease, and gastric cancer, wherein the method comprises:
(a) taking a saliva sample from an individual suspected of having a gastroesophageal disease of the specific gastroesophageal diseases;
(b) combining the saliva sample with sodium azide;
(c) testing the saliva sample to obtain measured levels of EGF, pepsin and one or more additional salivary biomarkers selected from a group consisting of E-cadherin, TGF-α, IL-6, MOB kinase activator 1A, EphA1, MOB kinase Activator 1B, integrin alpha 5, Golgi resident protein GCP 60, FR-beta, protein SEC13 homolog, epiregulin, FGF-12, inhibitor of nuclear factor kappa B kinase catalytic subunit, vimentin, annexin A1, protein S10-A6, malate dehydrogenase, proteasome activator complex subunit 2, cathepsin D light chain, MMP-1, MMP-2, MMP-3, MMP-7, MMP-8, MMP-9, MMP-10, MMP-13, MMP-14, mucin-1, alpha-1-acid glycoprotein, antithrombin, serpin peptidase inhibitor, CTSF, HMGB1, TLR7, COPS2, NT5E, TERF1, TIMP-1, TIMP-2, TIMP-4, XPNPEP2, TGFR2, SIGLEC6, CPE, GHR, GPNMB, SLAMF8, TNFRSF19, TWEAK, IFNGR1, Notch-3, TNFRSF19L, annexin A6, α-defensin-1, caveolin 1, EGF receptor, integrin beta 4, S100A6, S100A8, S100A9, and haptoglobin to create a first biomarker profile; and
(d) comparing the measured levels of the first biomarker profile with reference levels for the salivary biomarkers, wherein a decrease and/or increase in levels of the salivary biomarkers in the first biomarker profile relative to the reference levels is indicative of the gastroesophageal disease.

14. The method of claim 13, further comprising:
(e) if, based on the measured levels of the first biomarker profile relative to the reference levels the individual is determined to have the gastroesophageal disease, administering a treatment to the individual for the gastroesophageal disease, wherein the treatment includes one or more of antacids, H-2 receptor blockers, proton pump inhibitors, surgery, transoral incisionless fundoplication, a LINX device, fundoplication, prokinetics, acupuncture, surgical intervention, resection, and chemotherapy.

15. The method of claim 13, wherein the first biomarker profile with the measured levels meeting two or more criteria in a group of criteria is indicative of the gastroesophageal disease, the group of criteria consisting of:
measured levels of EGF, wherein EGF levels between 145 and 98.2 pg/mL is indicative of non-erosive reflux disease;
measured levels of pepsin, wherein pepsin levels between 184.6 and 151 ng/mL is indicative of erosive reflux disease; pepsin levels between 145 and 98.2 ng/mL are indicative of non-erosive reflux disease; and pepsin levels between 255.8-207.4 ng/mL are indicative of gastric cancer; and
measured levels of E-cadherin, wherein E-cadherin levels between 18.3-15.1 ng/mL are indicative of erosive reflux disease; E-cadherin levels between 14.9-11.5 ng/mL are indicative of non-erosive reflux disease; and E-cadherin levels of 42.5-38.3 ng/mL are indicative of gastric cancer.

16. The method of claim 13, wherein the decrease and/or increase in levels of the salivary biomarkers in the first biomarker profile relative to the reference levels is indicative of a specific stage of the gastroesophageal disease;
wherein measured levels of pepsin less than 147.15 ng/mL and E-cadherin of less than 11.5 ng/mL is indicative of mild gastroesophageal reflux disease.

17. The method of claim 13, further comprising (e) administering a gastrointestinal endoscopy examination to the individual if levels of any two or more salivary biomarkers of the first biomarker profile are within respective threshold ranges, wherein the threshold ranges are E-cadherin ≥5 ng/ml, EGF≥978 pg/mL, TGF-α≥19.2 pg/mL, pepsin ≥92.4 ng/mL, MMP-7≥724 pg/mL, MMP-2≥12.4 pg/mL, MMP-1≥78 pg/ml, MMP-3≥100 ng/ml, MMP-8≥245 ng/mL, MMP-9≥95 ng/ml, MMP-10≥90 pg/ml, MMP-13≥0.05 ng/ml, S100A8≥0.4 pg/ml, S100A9≥91 ng/ml, haptoglobin ≥15 μg/mL, TIMP-1≤291 pg/ml, and/or TIMP-2≤190 pg/ml.

18. The method of claim 13, further comprising (e) administering a gastrointestinal endoscopy examination to the individual if levels of any two or more salivary biomarkers of the first biomarker profile are within respective threshold ranges of respective target values, wherein the target values are E-cadherin 5 ng/ml, EGF 978 pg/mL, TGF-α 19.2 pg/mL, pepsin 92.4 ng/mL, MMP-7 724 pg/mL, MMP-2 12.4 pg/mL, MMP-1 78 pg/ml, MMP-3 100 ng/ml, MMP-8 245 ng/mL, MMP-9 95 ng/ml, MMP-10 90 pg/ml, MMP-13 0.05 ng/ml, S100A8 0.4 pg/ml, S100A9 91 ng/ml, haptoglobin 15 μg/mL, TIMP-1 291 pg/ml, and/or TIMP-2 190 pg/ml, and wherein each threshold range is plus and minus 10% of the respective target value.

19-20. (canceled)

21. The method of claim 14, wherein after the treatment has begun, a second saliva sample is collected from the individual, and the second saliva sample is tested for levels of salivary EGF, pepsin, and one or more of salivary E-cadherin, TGF-α, IL-6, MOB kinase activator 1A, EphA1, MOB kinase Activator 1B, integrin alpha 5, Golgi resident protein GCP 60, FR-beta, protein SEC13 homolog, epiregulin, FGF-12, inhibitor of nuclear factor kappa B kinase catalytic subunit, vimentin, annexin A1, protein S100-A6, malate dehydrogenase, proteasome activator complex subunit 2, cathepsin D light chain, MMP-1, MMP-2, MMP-3, MMP-7, MMP-8, MMP-9, MMP-10, MMP-13, MMP-14, mucin-1, alpha-1-acid glycoprotein, antithrombin, serpin peptidase inhibitor, CTSF, HMGB1, TLR7, COPS2, NT5E, TERF1, TIMP-1, TIMP-2, TIMP-4, XPNPEP2, TGFR2, SIGLEC6, CPE, GHR, GPNMB, SLAMF8, TNFRSF19, TWEAK, IFNGR1, Notch-3, TNFRSF19L, annexin A6, α-defensin-1, caveolin 1, EGF receptor, integrin beta 4, S100A6, S100A8, S100A9, and haptoglobin to create a second biomarker profile; and
the second biomarker profile is compared to the first biomarker profile, wherein a decrease and/or increase in levels of the salivary biomarkers in the second biomarker profile relative to the first biomarker profile indicates that the treatment is effective.

22. The method of claim 21, wherein the individual is treated with a proton pump inhibitor, wherein effective treatment with the proton pump inhibitor decreases EGF and pepsin levels in the second biomarker profile by at least 50% in comparison to the first biomarker profile.

23. A system for analysis of a saliva sample obtained from a patient suspected of suffering from a gastroesophageal disease selected from the group consisting of erosive reflux disease, non-erosive reflux disease, and gastric cancer, the system comprising a test strip and a reader;
wherein the test strip is configured to produce a detectable output at a level proportional to a level present on the test strip of each of EGF, pepsin and one or more additional salivary biomarkers, wherein the additional salivary biomarkers are selected from E-cadherin, TGF-α, IL-6, MOB kinase activator 1A, EphA1, MOB kinase Activator 1B, integrin alpha 5, Golgi resident protein GCP 60, FR-beta, protein SEC13 homolog, epiregulin, FGF-12, inhibitor of nuclear factor kappa B kinase catalytic subunit, vimentin, annexin A1, protein S100-A6, malate dehydrogenase, proteasome activator complex subunit 2, cathepsin D light chain, MMP-1, MMP-2, MMP-3, MMP-7, MMP-8, MMP-9, MMP-10, MMP-13, MMP-14, mucin-1, alpha-1-acid glycoprotein, antithrombin, serpin peptidase inhibitor, CTSF, HMGB1, TLR7, COPS2, NT5E, TERF1, TIMP-1, TIMP-2, TIMP-4, XPNPEP2, TGFR2, SIGLEC6, CPE, GHR, GPNMB, SLAMF8, TNFRSF19, TWEAK, IFNGR1, Notch-3, TNFRSF19L, annexin A6, α-defensin-1, caveolin 1, EGF receptor, integrin beta 4, S100A6, S100A8, S100A9, and haptoglobin;
wherein after the test strip is exposed to the saliva sample, the reader is configured to read the detectable output; and
wherein when the detectable output indicates that EGF, pepsin, and/or the one or more additional salivary biomarkers meet one or more criteria in a group of criteria, the gastroesophageal disease is indicated.

24. The system of claim 23, wherein the reader provides a qualitative, semi-quantitative, or quantitative measure of EGF, pepsin and the one or more additional salivary biomarkers.

25. The system of claim 23, wherein the detectable output is generated by a fluorescent dye, chemiluminescent compound, radioisotope, electron-dense reagent, enzyme, colored particle, nanoparticle, metal sol or colloid, gold or silver nanoparticle nanoparticle, colored latex bead, up-converting phosphor, magnetic particle, carbon nanoparticle, nanoparticles, quantum dots, or organic fluorophores.

26. The system of claim 24, wherein the reader is an optical detector.
